(19) United States
(12) Patent Application Publication
(10) Pub. No.: US 2016/0120742 A1
(43) Pub. Date: May 5, 2016

(54) COMPOSITIONS INCLUDING CABAZITAXEL

(71) Applicant: Sandoz AG, Basel (CH)

(72) Inventor: Priyanga Wickramanayake, Unterach (AT)

(21) Appl. No.: 14/532,703

(22) Filed: Nov. 4, 2014

(51) Int. Cl. A61J 1/00; A61K 45/06; A61K 47/10; A61K 47/12; A61K 31/337; A61K 47/26

(52) U.S. Cl. CPC A61J 1/00; A61K 31/337; A61K 47/26; A61K 47/10; A61K 47/12; A61K 45/06

(57) ABSTRACT

Provided herein are pharmaceutical compositions including cabazitaxel and parenteral formulations including cabazitaxel. The compositions and parenteral formulations are useful in methods for treating prostate cancer including administering the parenteral formulations prepared with the compositions described herein.

### TECHNICAL FIELD

Embodiments of the present invention relate to pharmaceutical compositions including cabazitaxel and parenteral formulations including cabazitaxel. The compositions and parenteral formulations are useful in methods for treating prostate cancer including administering the parenteral formulations prepared with the compositions.

### BACKGROUND

Cabazitaxel is an anti-neoplastic agent that is administered to the patient via intravenous injection or infusion. It is commercially available under the trade name Jevtana®. Jevtana® is sold as a two vial system, wherein the first vial contains a cabazitaxel sterile concentrate and the second vial contains the solvent for preparing a pre-mix solution. The pre-mix solution is then used to prepare the final parenteral formulation. One ml of concentrate contains 40 mg cabazitaxel. Each vial of 1.5 ml (nominal volume) concentrate contains 60 mg cabazitaxel. After initial dilution with the entire solvent, each ml of solution contains 10 mg cabazitaxel. Both Jevtana® 60 mg/1.5 ml concentrate vial (fill volume: 73.2 mg of cabazitaxel/1.83 ml) and the solvent vial (fill volume: 5.67 mL) contain an overfill to compensate for liquid loss during preparation. This overfill ensures that after dilution with the entire contents of the accompanying solvent, there is solution containing 10 mg/ml cabazitaxel. The concentrate vial contains in addition to cabazitaxel, polysorbate 80 and citric acid. The solvent vial contains ethanol and water. The shelf life of the unopened concentrate vial is reported to be 3 years.

Jevtana® requires two dilutions prior to intravenous infusion. The second vial (the solvent) of Jevtana® is a solution containing 13% (w/w) ethanol in water for injection. The pre-mix solution is prepared by first diluting the concentrate of the first vial with the entire contents of the second. This requires repeated inversions for at least 45 seconds to ensure complete mixing of the concentrated drug solution and the diluent. The pre-mix solution, having a concentration of 10 mg/ml cabazitaxel, should be used immediately, preferably within 30 minutes. A volume of pre-mix solution calculated based on a dose of 25 mg/m³ is withdrawn and injected into a PVC-free container of either 0.9% sodium chloride solution or 5% dextrose solution (also referred to as 5% glucose solution) for infusion. After the second dilution, the concentration of cabazitaxel in the prepared infusion solution should be between 0.10 mg/mL and 0.26 mg/mL. The prepared infusion solution should be used for intravenous administration immediately, or within 8 hours, if stored at room temperature, or within 24 hours, if stored under refrigerated conditions.

One problem of both the pre-mix and of the infusion solutions is that crystals and/or particulates may appear. In this case, the pre-mix or the infusion solution cannot be used and should be discarded. Additionally, the prescribing information for Jevtana® advises on the formation of foam during the preparation of the premix.

Hence, the commercially available Jevtana® formulation has a complex administration that involves many steps.

Patent application WO 2014/028704 A discloses one vial cabazitaxel formulations containing cabazitaxel, polysorbate 80, citric acid and ethanol. The vial is kept sealed in CO₂ atmosphere. The CO₂ atmosphere contributes to lowering the pH at certain ranges of pH values. A high amount of ethanol and/or polysorbate 80 is required to fill up the volume to 1 mL as they are the only liquid components of the formulation. The concentration of ethanol can reach 0.75 mL per mL of formulation. The high amount of ethanol may increase the instability of cabazitaxel. Additionally, it can present a problem for patients who are alcohol addicted, or suffer from liver diseases and epilepsy. The amount of polysorbate 80 can reach 800 mg/mL (for 10 mg/mL of cabazitaxel). High amounts of polysorbate 80 are believed to be associated with hypertension and the administration of a formulation containing a high amount of polysorbate may require slowing down the infusion rate to avoid a high concentration of polysorbate in the blood; see, e.g. Engels et al., Anti-Cancer Drugs (2007) 18:95-103.

Patent application CN 102068407 A discloses a cabazitaxel formulation containing cabazitaxel, polysorbate 80, citric acid and ethanol. A high amount of ethanol and/or polysorbate 80 is required to fill up the volume to 1 mL as they are the only liquid components of the formulation. The high amount of ethanol may increase the instability of cabazitaxel. Additionally, it can present a problem for patients who are alcohol addicted, or suffer from liver diseases and epilepsy. Further, as noted above, high amounts of polysorbate 80 are believed to be associated with hypertension and the administration of a formulation containing a high amount of polysorbate may require slowing down the infusion rate to avoid a high concentration of polysorbate in the blood.

Hence, there is the need for providing an improved formulation of cabazitaxel that is stable and allows for less complex preparation of the infusion to be administered to a patient and does not contain a high amount of polysorbate and/or alcohol.

Therefore, a problem underlying the present invention is the provision of a formulation of cabazitaxel that is stable, does not contain high amounts of polysorbate and alcohol, and/or involves only one step dilution, and hence, allows less complex preparation of a parenteral formulation including cabazitaxel to be administered to a patient.

### SUMMARY

Surprisingly, it was found that the issues discussed above can be solved by a pharmaceutical composition including:
a) from 0.5 to 1.5 weight-% of cabazitaxel or a pharmaceutically acceptable salt, solvate, or hydrate thereof, in some embodiments, cabazitaxel monohydrate or anhydrous cabazitaxel;
b) from 20 to 40 weight-% of at least one surfactant, in some embodiments the at least one surfactant is polyoxyethylene (20) sorbitan monooleate;
c) from 10 to 23 weight-% of at least one C₂-C₄ alcohol, in some embodiments the at least one C₂-C₄ alcohol is ethanol, and in some embodiments is absolute ethanol;
d) from 0.3 to 0.6 weight-% of at least one organic acid, in some embodiments the at least one organic acid is anhydrous citric acid;
e) from 40 to 60 weight-% of a vehicle, wherein the vehicle is a polyethylene glycol 300 in some embodiments; and
f) from 0 to 5 weight-% of water;
   in each case based on the total weight of the composition.

Such a pharmaceutical composition can be contained in a single vial, which is ready for use in that it only uses a single dilution into an infusion solution prior to administration to a patient.

The compositions and parenteral formulations described herein are useful in methods for treating prostate cancer including administering the parenteral formulations prepared with the compositions described herein.

### BRIEF DESCRIPTION OF THE DRAWINGS

FIG. 1: Representative HPLC spectrum using Method 2 of cabazitaxel composition: Batch B1-45 (as prepared in Examples 4 and 5) from vials after 6 months at 40° C./75% relative humidity (vial upright). The retention time of cabazitaxel was 10.8. The substances eluting after 12 minutes are due to other components of the formulation and are not impurities related to cabazitaxel.

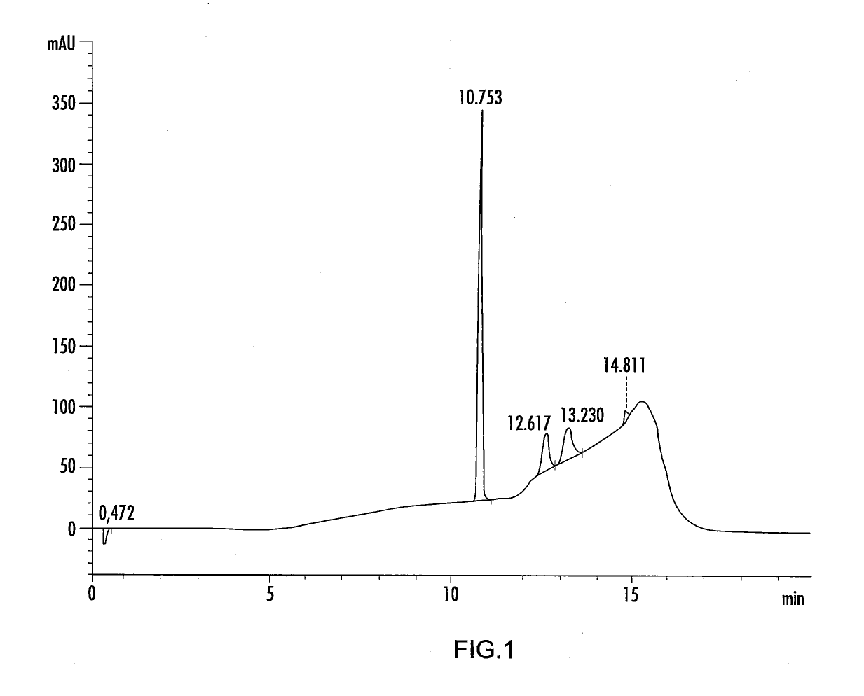

### DETAILED DESCRIPTION

Embodiments of the present invention are now described more fully. This invention may be embodied in many different forms and should not be construed as limited to the embodiments set forth herein; rather these embodiments are provided so that this disclosure will be thorough and complete and will fully convey the scope of the invention to those skilled in the art.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

As used herein, “weight-%” means the percentage of one component of the total mass of the composition or solution [(grams of solute/grams of solution)×100] and can also be abbreviated as “% (w/w)” or “w/w %”.

Formulation components reported in units of volume/volume are calculated by the volume of solute/volume of solution. This is referred to as volume percent or “% (v/v)” or “v/v %”.

Compositions and formulations may comprise, consist essentially of or consist of the components indicated as being contained or included in the compositions and formulations.

### a) Cabazitaxel

Cabazitaxel (CAS Registry Number: 183133-96-2) is an antimicrotubile antineoplastic agent belonging to the taxoid family and has formula (I):

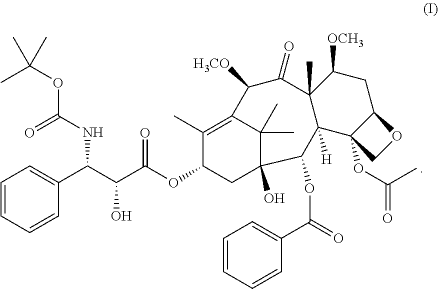 [SMILES: CO[C@H]1C(=O)[C@]2(C)[C@@H](OC)C[C@H]3OC[C@@]3(OC(C)=O)[C@H]2[C@H](OC(=O)c2ccccc2)[C@]2(O)C[C@H](OC(=O)[C@H](O)[C@@H](NC(=O)OC(C)(C)C)c3ccccc3)C(C)=C1C2(C)C]

It is a semi-synthetic taxoid derived from the major natural taxoid (10-deacety Baccatin III) extracted from the needles of various yew trees. Cabazitaxel acts by disrupting the microtubular network in cells that is essential for mitotic and interphase cellular functions. Like other taxanes, cabazitaxel binds to beta-tubulin subunits on microtubules and stabilizers and suppresses microtubule activity. This leads to the production of microtubules without normal function and to stabilization of microtubules which results in the inhibition of mitosis in cells.

The active pharmaceutical ingredient of the pharmaceutical composition is cabazitaxel or a pharmaceutically acceptable salt, solvate, or hydrate thereof. In some embodiments, cabazitaxel is cabazitaxel monohydrate or anhydrous cabazitaxel. In some embodiments, cabazitaxel is cabazitaxel monohydrate. Cabazitaxel or the pharmaceutically acceptable salt, solvate, or hydrate thereof is also referred to as the “active pharmaceutical ingredient” or “API”.

In some embodiments, the pharmaceutical composition contains cabazitaxel or the pharmaceutically acceptable salt, solvate, or hydrate thereof, in some embodiments, cabazitaxel is present at a concentration in the range of from 0.5 to 1.5 weight-%, in some embodiments, from 0.6 to 1.4 weight-%, in some embodiments, from 0.7 to 1.3 weight-%, in some embodiments, from 0.8 to 1.2 weight-%, in some embodiments, from 0.9 to 1.1 weight-%, and in some embodiments, in the range of from 0.92 to 1.01 weight-%, in each case based on the total weight of the composition.

In some embodiments, the pharmaceutical composition includes cabazitaxel or a pharmaceutically acceptable salt, solvate, hydrate thereof, in some embodiments cabazitaxel, at a concentration in the range of from 5 to 15 mg/mL, in some embodiments from 6 to 14 mg/mL, in some embodiments from 7 to 13 mg/mL, in some embodiments from 8 to 12 mg/ml, in some embodiments from 9 to 11 mg/ml, and in some embodiments from 9.5 mg/ml to 10.5 mg/ml. In some embodiments, at a temperature of the composition in the range of from 24 to 26° C. and a pressure in the range of from 0.95 to 1.05 bar, the pharmaceutical composition has a concentration of cabazitaxel in the range of from 5 to 15 mg/ml, in some embodiments from 6 to 14 mg/mL, in some embodiments from 7 to 13 mg/mL, in some embodiments from 8 to 12 mg/ml, in some embodiments from 9 to 11 mg/ml, and in some embodiments from 9.5 mg/ml to 10.5 mg/ml.

### b) Surfactant

The pharmaceutical composition contains at least one surfactant. Any surfactant suitable for solubilizing cabazitaxel is generally conceivable as surfactant. In some embodiments, the surfactant is used to solubilize cabazitaxel in the final parenteral formulation to be administered to a patient.

In some embodiments, surfactants have a dual hydrophobic/hydrophilic nature, hence in solution they tend to orient themselves so that the exposure of the hydrophobic portion of the surfactant to an aqueous solution, in particular the aqueous solution of the parenteral formulation, is minimized, thus forming micelles.

Without wanting to be bound to any theory it is assumed that API is solubilized being surrounded by a micelle. Hence, the amount of a surfactant in the pharmaceutical composition may be chosen such that the API is essentially completely solubilized. The concentration of the at least one surfactant in the pharmaceutical composition may be such that the concentration of the at least one surfactant in the parenteral formulation is equal or greater than the critical micelle concentration, also referred herein as CMC, of said at least one surfactant. The method of measuring the CMC is not particularly limited and may be measured by known methods, including, for example, the methods described in Das and Hajra, Journal of Biological Chemistry (1992) 167(14): 9731. In particular embodiments, the concentration of a given surfactant is above the CMC of said surfactant.

In some embodiments, the intended cabazitaxel concentration of the final infusion solution is about 0.10 to 0.26 mg/ml, and in some embodiments the concentration of cabazitaxel is about 0.18 mg/ml. In some embodiments, the infusion solution is about 0.9% NaCl or 5% glucose.

In some embodiments, the micelle system is stable over an infusion time of up to about 60 minutes as discussed in greater detail in Example 6.

Further, without wanting to be bound by any theory, it is believed that these exemplary concentrations of the at least one surfactant avoid the crystallization of the API in the pharmaceutical composition and the parenteral formulation.

According to particular embodiments, the at least one surfactant according to b) is a polysorbate, a poloxamer, a polyoxyglyceride, or a combination of two or more thereof, in some embodiments, a polysorbate or a combination of two or more thereof. In some embodiments, the at least one surfactant comprises or consists of, a polysorbate. In particular embodiments, the at least one surfactant comprises or consists of, polyoxyethylene (20) sorbitan monooleate. Polyoxyethylene (20) sorbitan monooleate is also known under its trademark name “Tween 80” or as “polysorbate 80”, and is assigned CAS Registry Number 9005-65-6.

In some embodiments, the pharmaceutical composition includes the at least one surfactant at a concentration in the range of from 20 to 40 weight-%, in some embodiments from 20 to 35 weight-%, in some embodiments from 20 to 30 weight-%, in some embodiments from 21 to 29 weight-%, in some embodiments from 22 to 28 weight-%, in some embodiments from 23 to 27 weight-%, in each case based on the total weight of the composition. If the composition includes more than one surfactant, the above concentration ranges refer to the concentration of the sum of all surfactants.

In some embodiments, the pharmaceutical composition includes the at least one surfactant at a concentration in the range of from 20 to 40 weight-%, in some embodiments of from 20 to 35 weight-%, in some embodiments from 20 to 30 weight-%, in some embodiments from 21 to 29 weight-%, in some embodiments from 22 to 28 weight-%, in some embodiments from 23 to 27 weight-%, in each case based on the total weight of the composition, wherein the at least one surfactant is at least one polysorbate. If the composition includes more than one surfactant, the above concentration ranges refer to the concentration of the sum of all surfactants.

In some embodiments, the pharmaceutical composition includes the at least one surfactant at a concentration in the range of from 20 to 40 weight-%, in some embodiments from 20 to 35 weight-%, in some embodiments from 20 to 30 weight-%, in some embodiments from 21 to 29 weight-%, in some embodiments from 22 to 28 weight-%, in some embodiments from 23 to 27 weight-%, in each case based on the total weight of the composition, wherein the at least one surfactant includes polyoxyethylene (20) sorbitan monooleate. If the composition includes more than one surfactant, the above concentration ranges refer to the concentration of the sum of all surfactants.

In some embodiments, the pharmaceutical composition includes the at least one surfactant at a concentration in the range of from 208 to 415 mg/mL, in some embodiments from 208 to 364 mg/mL, in some embodiments from 208 to 311 mg/mL, in some embodiments from 218 to 301 mg/mL, in some embodiments from 228 to 290 mg/mL, in some embodiments from 238 to 280 mg/mL, wherein the at least one surfactant includes polyoxyethylene (20) sorbitan monooleate. If the composition includes more than one surfactant, the above ranges refer to the concentration of the sum of all surfactants. In some embodiments, at a temperature of the composition in the range of from 24 to 26° C. and a pressure in the range of from 0.95 to 1.05 bar, the pharmaceutical composition includes the at least one surfactant at a concentration in the range of from 208 to 415 mg/mL, in some embodiments from 208 to 364 mg/mL, in some embodiments from 208 to 311 mg/mL, in some embodiments from 218 to 301 mg/mL, in some embodiments from 228 to 290 mg/mL, in some embodiments from 238 to 280 mg/mL, wherein the at least one surfactant includes polyoxyethylene (20) sorbitan monooleate. If the composition includes more than one surfactant, the above ranges refer to the concentration of the sum of all surfactants.

In some embodiments, the amount of the at least one surfactant is such that upon dilution in the final parenteral formulation, the amount complies with the requirements of the World Health Organization (WHO), the U.S. Food and Drug Administration (FDA) and similar European and National Offices; see, e.g. Nema and Brendel, PDA J. Pharm. Sci. and Tech. (2011) 65:287-332.

c) C₂-C₄ alcohol

The composition includes at least one C₂-C₄ alcohol. Generally, it is conceivable that the at least one alcohol may contain propylene glycol, benzyl alcohol, or a mixture thereof. Without wanting to be bound by any theory, it is believed that the at least one alcohol allows solubility of the API and/or avoidance of phase separation.

It was observed that when mixing together a component b) such as a polysorbate and component e) such as a polyethylene glycol at certain concentrations, these components may not be miscible, possibly leading to a phase separation. Such phase separation was visible already at a temperature of from 2 to 8° C. and sometimes still visible at a temperature of 40° C. However, phase separation of the components is detrimental for the homogenous distribution of the API in the composition and, thus, should be avoided. It has been surprisingly found that the addition of an alcohol, in some embodiments a C₂-C₄ alcohol, in some embodiments ethanol, and in some embodiments absolute ethanol, solves the problem of the phase separation, in particular the phase separation between the components b) and e) and renders the pharmaceutical composition homogeneous.

It is believed that the concentration of the at least one C₂-C₄ alcohol is relevant to ensure both the solubilization of the API and the miscibility of a component b), such as polysorbate, and a component e), such as polyethylene glycol. A too low amount of C₂-C₄ alcohol may not ensure solubilization of the API and the miscibility of the polysorbate and polyethylene glycol. On the other hand, it has been observed that the present composition is stable if the amount of C₂-C₄ alcohol is not too high. In particular, it has been observed that compositions including less than about 10 weight-% ethanol may show a phase separation, and that in compositions including more than about 23 weight-% ethanol, the API may degrade, as shown in Examples 1 to 3.

In some embodiments, the at least one C₂-C₄ alcohol is ethanol, propanol, isopropanol, butanol, 2-butanol, tert-butanol or a combination of two or more thereof, in some embodiments ethanol, isopropanol or a combination thereof, in some embodiments ethanol. In some embodiments, the ethanol is absolute ethanol wherein the term “absolute ethanol” or “ethanol (absolute)” refers to ethanol known in the art as “ethanol 99.8%”.

The at least one C₂-C₄ alcohol is present in an amount in the range of from 10 to 23 weight-%, in some embodiments from 14 to 22 weight-%, in some embodiments from 15 to 22 weight-%, in some embodiments from 16 to 22 weight-%, in some embodiments from 17 to 21 weight-%, in each case based on the total weight of the composition. If the composition includes more than one C₂-C₄ alcohol, the above concentration ranges refer to the concentration of the sum of all C₂-C₄ alcohol.

The at least one C₂-C₄ alcohol is present in an amount in the range of from 10 to 23 weight-%, in some embodiments from 14 to 22 weight-%, in some embodiments from 15 to 22 weight-%, in some embodiments from 16 to 22 weight-%, in some embodiments from 17 to 21 weight-%, in each case based on the total weight of the composition, wherein the at least one C₂-C₄ alcohol is ethanol, isopropanol or a combination thereof. If the composition includes more than one C₂-C₄ alcohol, the above concentration ranges refer to the concentration of the sum of all C₂-C₄ alcohol.

The at least one C₂-C₄ alcohol is present in an amount in the range of from 10 to 23 weight-%, in some embodiments from 14 to 22 weight-%, in some embodiments 15 to 22 weight-%, in some embodiments from 16 to 22 weight-%, in some embodiments from 17 to 21 weight-%, in each case based on the total weight of the composition, wherein the at least one C₂-C₄ alcohol includes, ethanol and in some embodiments absolute ethanol. If the composition includes more than one C₂-C₄ alcohol, the above concentration ranges refer to the concentration of the sum of all C₂-C₄ alcohol.

The at least one C₂-C₄ alcohol is present in an amount in the range of from 104 to 240 mg/mL, in some embodiments from 145 to 240 mg/mL, in some embodiments from 155 to 230 mg/mL, in some embodiments from 165 to 230 mg/mL, in some embodiments from 176 to 218 mg/mL, wherein the at least one C₂-C₄ alcohol includes ethanol and in some embodiments absolute ethanol. In some embodiments, at a temperature of the composition in the range of from 24 to 26° C. and a pressure in the range of from 0.95 to 1.05 bar, the at least one C₂-C₄ alcohol is present in an amount in the range of from 104 to 240 mg/mL, in some embodiments from 145 to 240 mg/mL, in some embodiments from 155 to 230 mg/mL, in some embodiments from 165 to 230 mg/mL, in some embodiments from 176 to 218 mg/mL wherein the at least one C₂-C₄ alcohol includes ethanol and in some embodiments absolute ethanol. If the composition includes more than one C₂-C₄ alcohol, the above concentration ranges refer to the concentration of the sum of all C₂-C₄ alcohol.

Instead of or in addition to the C₂-C₄ alcohol, propylene glycol and benzyl alcohol can be employed. The at least one C₂-C₄ alcohol or the propylene glycol or the benzyl alcohol or the mixture of two or more thereof is present in an amount in the range of from 10 to 23 weight-%, in some embodiments from 14 to 22 weight-%, in some embodiments from 15 to 22 weight-%, in some embodiments from 16 to 22 weight-%, in some embodiments from 17 to 21 weight-%, in each case based on the total weight of the composition. If the composition includes more than one of these alcohols, the above concentration ranges refer to the concentration of the sum of all of these alcohols.

The compositions are particularly advantageous since by balancing the amounts of the components b), c) and e) it is possible to obtain a stable cabazitaxel composition, wherein the cabazitaxel is optimally solubilized and the amount of alcohol is kept low. This is of major importance in particular if ethanol is used as component c) and the composition has to be administered to alcohol addicted patients or patients further suffering of liver diseases or epilepsy. Hence, the compositions are advantageous over the pharmaceutical compositions disclosed in patent applications WO 2014/028704 A and CN 102068407 A which contain a higher amount of ethanol.

### d) Organic Acid, pH

The pharmaceutical composition further includes at least one organic acid or mixture thereof. The acid may be any pharmaceutically acceptable organic acid known to the skilled person, which acids are soluble in a non-aqueous solvent system and are compatible with the API.

In some embodiments, the at least one organic acid is a weak acid. In some embodiments, the at least one organic acid is citric acid, tartaric acid, succinic acid, fumaric acid, and propionic acid, or any mixtures of two or more thereof. In some embodiments, the acid is citric acid, in some embodiments anhydrous citric acid or citric acid monohydrate, in some embodiments, anhydrous citric acid.

In some embodiments, the at least one organic acid is present in an amount in the range of from 0.01 to 1 weight-%, in some embodiments from 0.05 to 0.9 weight-%, in some embodiments from 0.1 to 0.8 weight-%, in some embodiments from 0.2 to 0.7 weight-%, in some embodiments from 0.3 to 0.6 weight-%, in some embodiments from 0.4 to 0.5 weight-%, in each case based on the total weight of the composition.

In some embodiments, the at least one organic acid is present in an amount in the range of from 0.3 to 0.6 weight-%, in some embodiments from 0.35 to 0.55 weight-%, in some embodiments 0.4 to 0.5 weight-%, in each case based on the total weight of the composition, wherein the at least one organic acid includes citric acid, and in some embodiments is anhydrous citric acid.

In some embodiments, the at least one organic acid is citric acid, and in some embodiments, anhydrous citric acid, and is present in an amount in the range of from 3 to 6 mg/mL, in some embodiments from 3.5 to 5.5 mg/mL, in some embodiments from 4 to 5 mg/mL. In some embodiments, at a temperature of the composition in the range of from 24 to 26° C. and a pressure in the range of from 0.95 to 1.05 bar, the at least one organic acid is present in an amount in the range of from 3 to 6 mg/mL, in some embodiments from 3.5 to 5.5 mg/mL, in some embodiments from 4 to 5 mg/mL, wherein the at least one organic acid includes citric acid, in some embodiments anhydrous citric acid.

Generally, the amount of the at least one organic acid will be limited by its solubility in the composition. In some embodiments, the amount of acid is such that the acid if solid is essentially completely dissolved in the composition. Further, the amount of the at least one organic acid will be further limited by the relative strength of the acid. In some embodiments, the at least one organic acid is present in the composition in an amount such that the pH of the composition is at most 4, in some embodiments from 2 to 4, in some embodiments from 2.2 to 3.9, in some embodiments from 2.4 to 3.8, in some embodiments from 2.6 to 3.7, in some embodiments from 2.7 to 3.6, in some embodiments from 2.8 to 3.5. The pH values relate to the pH of the composition diluted with purified water (deionized water) at a ratio of composition relative to water of 1:9, the pH value being determined with a pH meter comprising a pH sensitive glass electrode. For example, 1 ml of the composition is dilute with deionized water to a total volume of 10 ml. The pH of the resulting diluted aqueous solution is measured. Said method of determining the pH is described in detail in Method 1 in the section “Examples”. In some embodiments, the pH value is determined at a temperature of the diluted composition in the range of from 15 to 30° C., in some embodiments 20 to 25° C.

Alternatively, a “relative pH” of the composition may be determined directly by using, for example, an ethanol electrode such as the Et-OH Trode from Metrohm (6.0269.100); see, e.g. A Guide to pH Measurement: The Theory and Practice of Laboratory pH Applications (Mettler Toledo). In some embodiments, when measuring the relative pH of the composition using an ethanol probe, the pH is from 4.8 to 5.6, and in some embodiments, from 5.0 to 5.5.

### e) Vehicle

Component e) is a vehicle. The term “vehicle” refers to a medium or diluent in which the API is formulated. In particular, the vehicle is a pharmaceutical acceptable vehicle.

In some embodiments, the vehicle is a polyethylene glycol. Polyethylene glycols are also known as macrogol in the pharmaceutical industry. In some embodiments, polyethylene glycols have a mean molecular weight (Mw) in the range of from 200 to 35.000 Da. They are also referred to using the term “PEG” in combination with a numerical value such as 200, 300 or 400. The numerical value indicates the average molecular weight; thus, for example, PEG 200 is a polyethylene glycol having a mean molecular weight of 200 Da. In some embodiments, the polyethylene glycol is polyethylene glycol 200 (PEG 200), polyethylene glycol 300 (PEG 300), polyethylene glycol 400 (PEG 400) or a mixture of two or more thereof. In some embodiments, the polyethylene glycol includes polyethylene glycol 300 (PEG 300). In some embodiments, the vehicle is polyethylene glycol 300 (PEG 300).

In some embodiments, the vehicle is present in the pharmaceutical composition in an amount in the range of from 40 to 60 weight-%, in some embodiments from 45 to 60 weight-%, in some embodiments from 47 to 59 weight-%, in some embodiments from 49 to 58 weight-%, in some embodiments from 50 to 57 weight-%, in each case based on the total weight of the composition.

In some embodiments, the vehicle is present in the pharmaceutical composition in an amount in the range of from 40 to 60 weight-%, in some embodiments from 45 to 60 weight-%, in some embodiments from 47 to 59 weight-%, in some embodiments from 49 to 58 weight-%, in some embodiments from 50 to 57 weight-%, in each case based on the total weight of the composition, wherein the vehicle includes, in some embodiments, a polyethylene glycol.

In some embodiments, the vehicle is present in the pharmaceutical composition in an amount in the range of from 40 to 60 weight-%, in some embodiment, from 45 to 60 weight-%, in some embodiments from 47 to 59 weight-%, in some embodiments from 49 to 58 weight-%, in some embodiments from 50 to 57 weight-%, in each case based on the total weight of the composition, wherein the vehicle includes, in some embodiments, a polyethylene glycol 300.

In some embodiments, the vehicle is present in the pharmaceutical composition in an amount in the range of from 415 to 625 mg/mL, in some embodiments from 465 to 625 mg/mL, in some embodiments from 490 to 615 mg/mL, in some embodiments from 510 to 600 mg/mL, in some embodiments from 520 to 590 mg/mL, wherein the vehicle includes, in some embodiments, a polyethylene glycol 300. In some embodiments, at a temperature of the composition in the range of from 24 to 26° C. and a pressure in the range of from 0.95 to 1.05 bar, the vehicle is present in the pharmaceutical composition in an amount in the range of from 415 to 625 mg/mL, in some embodiments from 465 to 625 mg/mL, in some embodiments from 490 to 615 mg/mL, in some embodiments from 510 to 600 mg/mL, in some embodiments from 520 to 590 mg/mL, wherein the vehicle includes, in some embodiments, a polyethylene glycol 300.

In some embodiments of the pharmaceutical composition, polysorbate according to b) is polyoxyethylene(20) sorbitan monooleate, the C₂-C₄ alcohol according to c) is ethanol, in some embodiments absolute ethanol, the organic acid according to d) is citric acid, in some embodiments anhydrous citric acid, and the polyethylene glycol according to e) is polyethylene glycol 300.

In some embodiments, the pharmaceutical composition includes:
a) from 0.7 to 1.3 weight-% of the cabazitaxel or the pharmaceutically acceptable salt, solvate, or hydrate thereof, in some embodiments cabazitaxel monohydrate or anhydrous cabazitaxel;
b) from 20 to 30 weight-% of the at least one surfactant, wherein the at least one surfactant in some embodiments includes a polysorbate, in some embodiments includes polyoxyethylene(20) sorbitan monooleate;
c) from 15 to 22 weight-% of the at least one C₂-C₄ alcohol, wherein the at least one C₂-C₄ alcohol in some embodiments includes ethanol, in some embodiments absolute ethanol;
d) from 0.3 to 0.6 weight-% of the at least one organic acid, wherein the at least one organic acid in some embodiments includes citric acid, in some embodiments anhydrous citric acid;
e) from 45 to 60 weight-% of the vehicle, wherein the vehicle in some embodiments includes a polyethylene glycol, wherein the vehicle in some embodiments is polyethylene glycol 300;
f) from 0 to 3 weight-% of water;
   in each case based on the total weight of the composition.

In some embodiments, the pharmaceutical composition includes:
a) from 0.9 to 1.1 weight-% of the cabazitaxel or the pharmaceutically acceptable salt, solvate, or hydrate thereof, in some embodiments cabazitaxel monohydrate or anhydrous cabazitaxel;
b) from 23 to 27 weight-% of the at least one surfactant, wherein the at least one surfactant in some embodiments is a polysorbate, and in some embodiments is polyoxyethylene(20) sorbitan monooleate;
c) from 17 to 21 weight-% of the at least one C₂-C₄ alcohol, wherein the at least one C₂-C₄ alcohol in some embodiments is ethanol, and in some embodiments is absolute ethanol;
d) from 0.35 to 0.55 weight-% of the at least one organic acid, wherein the at least one organic acid in some embodiments is citric acid, and in some embodiments anhydrous citric acid;
e) from 50 to 57 weight-% of the vehicle, wherein the vehicle in some embodiments is a polyethylene glycol, wherein the vehicle in some embodiments is polyethylene glycol 300;
f) from 0 to 1 weight-% of water;
   in each case based on the total weight of the composition.

In some embodiments, at least 95 weight-%, in some embodiments at least 96 weight-%, in some embodiments at least 97 weight-%, in some embodiments at least 98 weight-%, in some embodiments at least 99 weight-%, of the pharmaceutical composition described above includes components a), b), c) d), and e). In some embodiments, at least 99.5 weight-%, in some embodiments at least 99.7 weight-% of the pharmaceutical composition described above includes components a), b), c) d), and e).

Therefore, in some embodiments, the pharmaceutical composition includes:

a) from 0.9 to 1.1 weight-% of cabazitaxel;
b) from 23 to 27 weight-% of polyoxyethylene(20) sorbitan monooleate;
c) from 17 to 21 weight-% of ethanol;
d) from 0.3 to 0.6 weight-% of citric acid;
e) from 50 to 57 weight-% of polyethylene glycol;
f) from 0 to 1 weight-% of water;
in each case based on the total weight of the composition, wherein at least 99.5 weight-% of the pharmaceutical composition includes components a), b), c), d), and e).

In some embodiments the pharmaceutical composition includes:
a) from 0.9 to 1.1 weight-% of cabazitaxel, in some embodiments cabazitaxel monohydrate; in some embodiments 0.92 to 1.01 weight-% of cabazitaxel, in some embodiments cabazitaxel monohydrate
b) from 23 to 27 weight-% of polyoxyethylene(20) sorbitan monooleate;
c) from 17 to 21 weight-% of absolute ethanol;
d) from 0.4 to 0.5 weight-% of anhydrous citric acid;
e) from 50 to 57 weight-% of polyethylene glycol 300;
f) from 0 to 1 weight-% of water;
   in each case based on the total weight of the composition, wherein at least 99.7 weight-% of the pharmaceutical composition includes components a), b), c), d), and e).

In some embodiments, the pharmaceutical composition described above is liquid at a temperature in the range of from 15 to 30° C., in some embodiments from 20 to 30° C., in some embodiments from 24 to 26° C., at a pressure in the range of from 0.95 to 1.05 bar.

### Process for Preparing the Pharmaceutical Composition

Generally, no specific restrictions exist how the pharmaceutical composition is prepared or compounded. In some embodiments, the pharmaceutical composition is prepared by a process including mixing at least one C₂-C₄ alcohol, wherein in some embodiments the C₂-C₄ alcohol includes ethanol, in some embodiments the C₂-C₄ alcohol is ethanol and wherein the ethanol in some embodiments is absolute ethanol; with at least one organic acid, wherein the organic acid in some embodiments includes citric acid, in some embodiments the organic acid is citric acid and wherein the citric acid in some embodiments is anhydrous citric acid; cabazitaxel or the pharmaceutically acceptable salt, solvate, or hydrate thereof, in some embodiments cabazitaxel monohydrate or anhydrous cabazitaxel, at least one surfactant, wherein the surfactant in some embodiments is a polysorbate, in some embodiments a polysorbate and wherein the polysorbate is in some embodiments polyoxyethylene (20) sorbitan monooleate; and the vehicle, wherein the vehicle is in some embodiments a polyethylene glycol, wherein in some embodiments the vehicle is polyethylene glycol 300.

In some embodiments, the above disclosed process includes:
(i) dissolving the at least one organic acid in the at least one C₂-C₄ alcohol, obtaining a mixture I;
(ii) dissolving the cabazitaxel or the pharmaceutically acceptable salt, solvate, or hydrate thereof, preferably cabazitaxel monohydrate or anhydrous cabazitaxel, in the mixture I, obtaining a mixture II;
(iii) dissolving the at least one surfactant in the mixture II, obtaining a mixture III;
(iv) adding the vehicle to the mixture III, obtaining a mixture IV;
(v) mixing the mixture IV, obtaining the composition.

In some embodiments, the steps (i) to (v) are carried out at a temperature in the range of from 15 to 30° C., in some embodiments from 20 to 25° C.

In some embodiments, after the addition of each component, the composition is mixed for a sufficient length of time to obtain a homogenous solution.

In some embodiments, the preparation of the pharmaceutical composition is carried out under inert atmosphere, such as an argon atmosphere or a nitrogen atmosphere, in some embodiments a nitrogen atmosphere.

In some embodiments, after all the components of the pharmaceutical composition have been added, the composition is purged with an inert gas, such as argon gas or nitrogen gas, in some embodiments, nitrogen gas. After purging, the pharmaceutical composition includes less than 5% dissolved oxygen, in some embodiments less than 4% dissolved oxygen, in some embodiments less than 3% dissolved oxygen. The method of measuring the dissolved oxygen in the pharmaceutical composition is not particularly limited and may be measured according to known methods. For example, the dissolved oxygen content of the pharmaceutical composition can be measured by an amperometric oxygen sensor. For example, the dissolved oxygen content of the pharmaceutical composition can be measured by InPro6000 (Mettler Toledo).

In some embodiments, the pharmaceutical composition is a one vial formulation. The term “one vial formulation” means that the composition can be directly diluted to the final formulation to be administered to the patient, without the need of a pre-dilution. In contrast to two vial formulations such as Jevtana®, the pharmaceutical composition is stable and ready to be diluted with a parenteral infusion solution. This “one vial” feature is one of the major advantages of the composition over the two vial formulations, since it avoids cumbersome pre-dilution to prepare the pre-mix solution. Further, said pre-mix solutions, according to the packaging information, should also be used within a short time, for example 30 minutes, after the preparation. Hence, the pharmaceutical composition of is further advantageous because it avoids the problem of the immediate use of a premix solution.

Generally, the amount of composition in a given vial is not subject to any specific restrictions. Typically, a vial contains the composition including from 20 to 90 mg, in some embodiments from 22 to 85 mg, in some embodiments from 24 to 80 mg, in some embodiments from 26 to 75 mg, in some embodiments from 28 to 70 mg of cabazitaxel or the pharmaceutically acceptable salt, solvate, or hydrate thereof, in some embodiments cabazitaxel monohydrate or anhydrous cabazitaxel are included in the vial. In some embodiments, a vial includes the composition including from 30 to 65 mg of cabazitaxel or the pharmaceutically acceptable salt, solvate, or hydrate thereof, in some embodiments cabazitaxel monohydrate or anhydrous cabazitaxel are included in the vial. In some embodiments, a vial includes the composition including from 35 to 55 mg, in some embodiments from 40 to 50 mg, from 42 to 48 mg, or from 50 to 70 mg, in some embodiments from 55 to 65 mg, in some embodiments from 57 to 63 mg of cabazitaxel or the pharmaceutically acceptable salt, solvate, or hydrate thereof, in some embodiments cabazitaxel monohydrate or anhydrous cabazitaxel is included in the vial.

The vial is in some embodiments a glass vial. The vial is in some embodiments an air-tight vial. The vial is in some embodiments provided with a stopper which is in some embodiments an air-tight stopper. Rubber stoppers may be used for the purpose of providing an air-tight vial. By way of example, West chlorobutyl rubber with fluorotec coating stoppers can be used.

In some embodiments, the atmosphere in the headspace of the vial is an inert atmosphere, such as an argon atmosphere or a nitrogen atmosphere, in some embodiments a nitrogen atmosphere. Hence, the headspace of the vial may be filled or flushed with nitrogen gas prior to insertion of the stopper to provide an inert atmosphere.

The vial is in some embodiments an air-tight vial including an air-tight stopper. The headspace of the vial includes less than 3% oxygen, in some embodiments less than 2% oxygen, in some embodiments less than 1% oxygen. The method of measuring the oxygen content of the headspace of the vial is not particularly limited and may be measured according to known methods. The oxygen content of the headspace of the vial can be measured, for example, by a system using a near-infrared laser tuned to match an internal frequency of oxygen such as the Lighthouse Instruments Headspace Oxygen Analyzer Model FMS-760.

### One Vial Formulation

As mentioned above, “one vial” pharmaceutical composition means that the composition can be directly diluted to the final parenteral infusion solution, which is to be administered to the patient, without the need of a pre-dilution that is typical of two vial formulations such as Jevtana®.

A one vial pharmaceutical composition comprises, consists essentially of, or consists of:
a) from 0.9 to 1.1 weight-% of the cabazitaxel or the pharmaceutically acceptable salt, solvate, or hydrate thereof, in some embodiments cabazitaxel monohydrate or anhydrous cabazitaxel;
b) from 23 to 27 weight-% of the at least one surfactant, wherein the at least one surfactant in some embodiments includes a polysorbate, in some embodiments includes polyoxyethylene(20) sorbitan monooleate;
c) from 17 to 21 weight-% of the at least one C₂-C₄ alcohol, wherein the at least one C₂-C₄ alcohol in some embodiments includes ethanol, and in some embodiments is absolute ethanol;
d) from 0.35 to 0.55 weight-% of the at least one organic acid, wherein the at least one organic acid in some embodiments includes citric acid, and in some embodiments is anhydrous citric acid;
e) from 50 to 57 weight-% of the vehicle, wherein the vehicle in some embodiments is a polyethylene glycol, wherein the vehicle in some embodiments is polyethylene glycol 300;
f) from 0 to 1 weight-% of water;
   in each case based on the total weight of the composition, wherein the one vial pharmaceutical composition has a total volume of at least 4.5 ml, in some embodiments from 4.6 to 5.4 ml or from 6.2 to 7.0 ml. The volume of from 6.2 to 7.0 ml or from 4.6 to 5.4 ml, reflects a vial providing about 60 mg of API or about 45 mg of API, respectively. In some embodiments, the pharmaceutical composition includes less than 5% dissolved oxygen, in some embodiments less than 4% dissolved oxygen, in some embodiments less than 3% dissolved oxygen. In some embodiments, the vial is an air-tight vial including an air-tight stopper and including less than 3% oxygen in the headspace, in some embodiments less than 2% oxygen in the headspace, in some embodiments less than 1% oxygen in the headspace.

In some embodiments, a one vial pharmaceutical composition comprises, consists essentially of, or consists of:
a) from 0.9 to 1.1 weight-% of cabazitaxel, in some embodiments cabazitaxel monohydrate;
b) from 23 to 27 weight-% of polyoxyethylene(20) sorbitan monooleate;
c) from 17 to 21 weight-% of ethanol;
d) from 0.3 to 0.6 weight-% of citric acid;
e) from 50 to 57 weight-% of polyethylene glycol;
f) from 0 to 1 weight-% of water;
   in each case based on the total weight of the composition, wherein at least 99.5 weight-% of the pharmaceutical composition includes components a), b), c) d), and e) and wherein the one vial pharmaceutical composition has a total volume of at least 4.5 ml, in some embodiments from 4.6 to 5.4 ml or from 6.2 to 7.0 ml. The volume of from 6.2 to 7.0 ml or from 4.6 to 5.4 ml, reflects a vial providing about 60 mg of API or about 45 mg of API, respectively. In some embodiments, the pharmaceutical composition includes less than 5% dissolved oxygen, in some embodiments less than 4% dissolved oxygen, in some embodiments less than 3% dissolved oxygen. In some embodiments, the vial is an air-tight vial including an air-tight stopper and including less than 3% oxygen in the headspace, in some embodiments less than 2% oxygen in the headspace, in some embodiments less than 1% oxygen in the headspace.

In some embodiments, a one vial pharmaceutical composition comprises, consists essentially of, or consists of:
a) from 0.9 to 1.1 weight-% of cabazitaxel, in some embodiments cabazitaxel monohydrate; in some embodiments 0.92 to 1.01 weight-% of cabazitaxel, in some embodiments cabazitaxel monohydrate;
b) from 23 to 27 weight-% of polyoxyethylene(20) sorbitan monooleate;
c) from 17 to 21 weight-% of absolute ethanol;
d) from 0.35 to 0.55 weight-% of anhydrous citric acid, in some embodiments 0.4 to 0.5 weight-% of anhydrous citric acid;
e) from 50 to 57 weight-% of polyethylene glycol 300;
f) from 0 to 1 weight-% of water;
   in each case based on the total weight of the composition, wherein at least 99.7 weight-% of the pharmaceutical composition includes components a), b), c) d), and e), and wherein the one vial pharmaceutical composition has a total volume of at least 4.5 ml, in some embodiments from 4.6 to 5.4 ml or from 6.2 to 7.0 ml. The volume of from 6.2 to 7.0 ml or from 4.6 to 5.4 ml, reflects a vial providing about 60 mg of API or about 45 mg of API, respectively. In some embodiments, the pharmaceutical composition includes less than 5% dissolved oxygen, in some embodiments less than 4% dissolved oxygen, in some embodiments less than 3% dissolved oxygen. In some embodiments, the vial is an air-tight vial including an air-tight stopper and including less than 3% oxygen in the headspace, in some embodiments less than 2% oxygen in the headspace, in some embodiments less than 1% oxygen in the headspace.

In some embodiments, a one vial pharmaceutical composition comprises, consists essentially of or consists of:
a) from 0.92 to 1.01 weight-% of cabazitaxel monohydrate;
b) from 23 to 27 weight-% of polyoxyethylene(20) sorbitan monooleate;
c) from 17 to 21 weight-% of absolute ethanol;
d) from 0.4 to 0.5 weight-% of anhydrous citric acid;
e) from 50 to 57 weight-% of polyethylene glycol 300;
f) from 0 to 1 weight-% of water;
   in each case based on the total weight of the composition, wherein at least 99.7 weight-% of the pharmaceutical composition includes components a), b), c) d), and e), and wherein the one vial pharmaceutical composition has a total volume of at least 4.5 ml, in some embodiments from 4.6 to 5.4 ml or from 6.2 to 7.0 ml. The volume of from 6.2 to 7.0 ml or from 4.6 to 5.4 ml, reflects a vial providing about 60 mg of API or about 45 mg of API, respectively. In some embodiments, the pharmaceutical composition includes less than 5% dissolved oxygen, in some embodiments less than 4% dissolved oxygen, in some embodiments less than 3% dissolved oxygen. In some embodiments, the vial is an air-tight vial including an air-tight stopper and including less than 3% oxygen in the headspace, in some embodiments less than 2% oxygen in the headspace, in some embodiments less than 1% oxygen in the headspace.

In some embodiments, a one vial pharmaceutical composition comprises, consists essentially of or consists of:
a) from 9.5 to 10.5 mg/ml of cabazitaxel or a pharmaceutically acceptable salt, solvate, or hydrate thereof, in some embodiments cabazitaxel monohydrate or cabazitaxel anhydrous, in some embodiments cabazitaxel monohydrate;
b) from 238 to 280 mg/ml of at least one surfactant, wherein the at least one surfactant in some embodiments is a polysorbate, in some embodiments is polyoxyethylene(20) sorbitan monooleate;
c) from 176 to 218 mg/ml of at least one C₂-C₄ alcohol, wherein the at least one C₂-C₄ alcohol in some embodiments is ethanol, and in some embodiments is absolute ethanol;
d) from 4 to 5 mg/ml of at least one organic acid, wherein the at least one organic acid in some embodiments is citric acid, and in some embodiments is anhydrous citric acid;
e) from 520 to 590 mg/ml of a vehicle, wherein the vehicle in some embodiments includes a polyethylene glycol, wherein the vehicle in some embodiments is polyethylene glycol 300;
f) from 0 to 1 mg/ml of water;
   wherein the one vial pharmaceutical composition has a total volume of at least 4.5 ml, in some embodiments from 4.6 to 5.4 ml or from 6.2 to 7.0 ml. The volume of from 6.2 to 7.0 ml or from 4.6 to 5.4 ml, reflects a vial providing about 60 mg of API or about 45 mg of API, respectively. In some embodiments, the pharmaceutical composition includes less than 5% dissolved oxygen, in some embodiments less than 4% dissolved oxygen, in some embodiments less than 3% dissolved oxygen. In some embodiments, the vial is an air-tight vial including an air-tight stopper and including less than 3% oxygen in the headspace, in some embodiments less than 2% oxygen in the headspace, in some embodiments less than 1% oxygen in the headspace.

In some embodiments, the one-vial pharmaceutical composition comprises, consists essentially of or consists of:
a) from 9.5 to 10.5 mg/ml of cabazitaxel monohydrate;
b) from 238 to 280 mg/ml of at least one surfactant, wherein the at least one surfactant is polyoxyethylene(20) sorbitan monooleate;
c) from 176 to 218 mg/ml of at least one C₂-C₄ alcohol, wherein the at least one C₂-C₄ alcohol is absolute ethanol;
d) from 4 to 5 mg/ml of at least one organic acid, wherein the at least one organic acid is anhydrous citric acid;
e) from 520 to 590 mg/ml of a vehicle, wherein the vehicle is polyethylene glycol 300;
f) from 0 to 1 mg/ml of water;
   wherein the one vial pharmaceutical composition has a total volume of at least 4.5 ml, in some embodiments from 4.6 to 5.4 ml or from 6.2 to 7.0 ml. The volume of from 6.2 to 7.0 ml or from 4.6 to 5.4 ml, reflects a vial providing about 60 mg of API or about 45 mg of API, respectively. In some embodiments, the pharmaceutical composition includes less than 5% dissolved oxygen, in some embodiments less than 4% dissolved oxygen, in some embodiments less than 3% dissolved oxygen. In some embodiments, the vial is an air-tight vial including an air-tight stopper and including less than 3% oxygen in the headspace, in some embodiments less than 2% oxygen in the headspace, in some embodiments less than 1% oxygen in the headspace.

### Parenteral Formulation

Regarding the parenteral fluid, no specific restrictions exist. In some embodiments, the parenteral fluid is a saline solution, a glucose solution, a dextran solution, or a mixture of two or more thereof. In some embodiments, the saline solution is a saline solution comprising 0.75 to 1.00 weight-% of sodium chloride. In some embodiments, the saline solution is a saline solution containing 0.9 weight-% of sodium chloride or is a normal saline solution. In some embodiments, it is a sterile solution. In some embodiments, the glucose solution is a solution including from 4 to 6 weight-% of glucose. In some embodiments, the glucose solution is a solution including 5 weight-% of glucose. In some embodiments, it is a sterile solution. In some embodiments, the dextran solution is a solution including from 4 to 6 weight-% of dextran. In some embodiments, it is a sterile solution.

This parenteral formulation results from the combination of the pharmaceutical composition and a parenteral fluid as indicated above, and the parenteral formulation obtained from this combination is ready-to-use for the administration to a patient in need thereof.

In some embodiments, the parenteral formulation includes the API in a concentration in the range of from 0.08 to 0.30 mg/ml, in some embodiments from 0.09 to 0.28 mg/ml, in some embodiments from 0.1 to 0.26 mg/ml. Therefore, the pharmaceutical composition or the one-vial pharmaceutical composition is admixed with an amount of parenteral fluid such that the final desired concentration of cabazitaxel in the final parenteral formulation is obtained.

In some embodiments, the parenteral formulation comprises, consists essentially of or consists of:
a) from 0.10 to 0.26 mg/ml of cabazitaxel or a pharmaceutically acceptable salt, solvate, or hydrate thereof, in some embodiments cabazitaxel monohydrate or anhydrous cabazitaxel;
b) from to 2.6 to 6.76 mg/ml of at least one surfactant, wherein the at least one surfactant in some embodiments is a polysorbate, and in some embodiments polyoxyethylene(20) sorbitan monooleate;
c) from 1.98 to 5.15 mg/ml of at least one C₂-C₄ alcohol, wherein the at least one C₂-C₄ alcohol in some embodiments is ethanol, and in some embodiments is absolute ethanol;
d) from 0.045 to 0.117 mg/ml of at least one organic acid, wherein the at least one organic acid is in some embodiments citric acid, and in some embodiments anhydrous citric acid;
e) from 5.60 to 14.6 mg/ml of a vehicle, wherein the vehicle in some embodiments includes a polyethylene glycol, wherein the vehicle in some embodiments is polyethylene glycol 300;
f) water to reach the desired volume.

In some embodiments, the parenteral formulation comprises, consists essentially of, or consists of:
a) from 0.10 to 0.26 mg/ml of cabazitaxel monohydrate or anhydrous cabazitaxel, in some embodiments cabazitaxel monohydrate;
b) from to 2.6 to 6.76 mg/ml of polyoxyethylene(20) sorbitan monooleate;
c) from 1.98 to 5.15 mg/ml of absolute ethanol;
d) from 0.045 to 0.117 mg/ml of anhydrous citric acid;
e) from 5.60 to 14.6 mg/ml of polyethylene glycol 300;
f) water to reach the desired volume.

The parenteral formulation is in some embodiments an intravenous formulation.

In some embodiments, the parenteral formulations are obtained or obtainable by a process including:
A) providing the pharmaceutical composition herein described;
B) admixing the composition provided according to A) with a parenteral fluid in some embodiments being a saline solution, a glucose solution, a dextran solution, or a mixture of two or more thereof;
   wherein the parenteral formulation includes from 0.10 to 0.26 mg/ml of cabazitaxel.

In some embodiments the parenteral formulation is a formulation wherein according to A), the pharmaceutical composition is provided via a vial including the pharmaceutical composition.

Generally, the preparation of the parenteral formulation is carried out in hospital at room temperature.

### Formulation for Use

In some embodiments, the parenteral formulation is a formulation for use as a medicament. In some embodiments, the parenteral formulation is a formulation for use in the treatment of a human or an animal. In some embodiments, the parenteral formulation is a formulation for use in the treatment of cancer, in some embodiments, prostate cancer. In some embodiments, the parenteral formulation for use is administered as an intravenous infusion for the required period of time. The parenteral formulation can be administered for the treatment of prostate cancer to a patient in need thereof. The parenteral formulation for use can be administered in combination with prednisone or prednisolone, wherein cabazitaxel and prednisone or prednisolone may be administered contemporaneously or sequentially.

### Method of Administering Cabazitaxel Compositions

The pharmaceutical composition, in particular the parenteral formulation prepared from the pharmaceutical composition is administered to a patient in need thereof. The parenteral formulation can be administered as intravenous infusion for the required period of time. The parenteral formulation can be administered to a patient in need thereof for the treatment of prostate cancer. The method may further include administering the parenteral formulation in combination with prednisone or prednisolone, wherein cabazitaxel and prednisone or prednisolone may be administered contemporaneously or sequentially.

### Stability of the Pharmaceutical Composition

The pharmaceutical composition is considered stable, if after storage for at least 3 months, in some embodiments, at least 6 months, the amount of cabazitaxel in the composition is at least 90%, in some embodiments, at least 92%, in some embodiments, at least 95%, of the initial amount of cabazitaxel in the composition at the start of storage. In some embodiments, the pharmaceutical composition is considered stable, if after at least 3 months, in some embodiments, at least 6 months, storage at 2-8° C., 25° C./60% relative humidity, 30° C./75% relative humidity, or 40° C./75% relative humidity, the amount of cabazitaxel in the composition is at least 90%, in some embodiments, at least 92%, in some embodiments, at least 95%, of the initial amount of cabazitaxel in the composition at the start of storage.

The stability of the pharmaceutical composition has been measured with methods known in the art and according to the United States Pharmacopeia (USP) and/or the European Pharmacopoeia (Ph. Eur.). The details of the methods are described herein below in the section “Examples”. Stability shelf parameters have been selected, which include the clarity, the pH, the total impurity content, the cabazitaxel identity, the cabazitaxel concentration and the ethanol concentration. The lack of variation with respect to the shelf parameters under different temperature and humidity conditions have been assessed after a pre-determined period of time, ranging from 1 to 6 months. It was found that the pharmaceutical composition is stable as shown by the results reported in Examples 1 to 6.

The following are illustrative embodiments and combinations of embodiments wherein in some embodiments, the pharmaceutical compositions include:
a) from 0.5 to 1.5 weight-% of cabazitaxel or a pharmaceutically acceptable salt, solvate, or hydrate thereof, in some embodiments cabazitaxel monohydrate or anhydrous cabazitaxel;
b) from 20 to 40 weight-% of at least one surfactant;
c) from 10 to 23 weight-% of at least one C₂-C₄ alcohol;
d) from 0.3 to 0.6 weight-% of at least one organic acid;
e) from 40 to 60 weight-% of a vehicle;
f) from 0 to 5 weight-% of water;
in each case based on the total weight of the composition.

In some embodiments, the pharmaceutical compositions described above include:
a) from 0.5 to 1.5 weight-% of cabazitaxel or a pharmaceutically acceptable salt, solvate, or hydrate thereof, in some embodiments cabazitaxel monohydrate or anhydrous cabazitaxel;
b) from 20 to 40 weight-% of at least one surfactant;
c) from 15 to 22 weight-% of at least one C₂-C₄ alcohol;
d) from 0.3 to 0.6 weight-% of at least one organic acid;
e) from 40 to 60 weight-% of a vehicle;
f) from 0 to 5 weight-% of water;
in each case based on the total weight of the composition.

In some embodiments described above, the compositions include:
a) from 0.7 to 1.3 weight-% of the cabazitaxel or the pharmaceutically acceptable salt, solvate, or hydrate thereof, in some embodiments cabazitaxel monohydrate or anhydrous cabazitaxel;
b) from 20 to 30 weight-% of the at least one surfactant;
c) from 15 to 22 weight-% of the at least one C₂-C₄ alcohol;
d) from 0.3 to 0.6 weight-% of the at least one organic acid;
e) from 45 to 60 weight-% of the at least one vehicle;
f) from 0 to 3 weight-% of water;
in each case based on the total weight of the composition.

In some embodiments, the compositions described above include:
a) from 0.92 to 1.01 weight-% of the cabazitaxel or the pharmaceutically acceptable salt, solvate, or hydrate thereof, in some embodiments cabazitaxel monohydrate or anhydrous cabazitaxel;
b) from 23 to 27 weight-% of the at least one surfactant;
c) from 17 to 21 weight-% of the at least one C₂-C₄ alcohol;
d) from 0.4 to 0.5 weight-% of the at least one organic acid;
e) from 50 to 57 weight-% of the at least one vehicle;
f) from 0 to 1 weight-% of water;
in each case based on the total weight of the composition.

In some embodiments of the compositions described above at least 95 weight-%, in some embodiments at least 97 weight-% of the compositions include components a), b), c), d), and e). In some embodiments, at least 99 weight-% of the composition includes components a), b), c), d), and e).

In some embodiments, the compositions described above can be a liquid at a temperature in the range of from 24 to 26° C. and a pressure in the range of from 0.95 to 1.05 bar.

In some embodiments, the compositions described above have a concentration of cabazitaxel or the pharmaceutically acceptable salt, solvate, or hydrate thereof, in some embodiments cabazitaxel monohydrate or anhydrous cabazitaxel, in the range of from 5 to 15 mg/ml, in some embodiments from 7 to 13 mg/ml, in some embodiments from 8 to 12 mg/ml, in some embodiments from 9 to 11 mg/ml, in some embodiments from 9.5 to 10.5 mg/ml. In some embodiments, the concentration of cabazitaxel monohydrate is in the range of from 9 to 11 mg/ml.

In some embodiments of the compositions described above, at least one surfactant according to b) is a polysorbate. In some embodiments, the at least one surfactant according to b) includes polyoxyethylene (20) sorbitan monooleate.

In some embodiments of the compositions described above the at least one C₂-C₄ alcohol according to c) is ethanol, propanol, isopropanol, butanol, isobutanol, tert-butanol, or a combination of two or more thereof, in some embodiments ethanol, isopropanol, or a combination thereof. In some embodiments, the at least one C₂-C₄ alcohol according to c) includes ethanol. In some embodiments, the ethanol is absolute ethanol.

In some embodiments of the compositions described above the at least one organic acid according to d) is a carboxylic acid or combination of two or more thereof, in some embodiments citric acid, ascorbic acid, tartaric acid, or a combination of two or more thereof. In some embodiments, the at least one organic acid according to d) comprises, in some embodiments consists of, citric acid. In some embodiments, the citric acid is anhydrous citric acid.

In some embodiments of the compositions described above the at least one vehicle according to e) is a polyethylene glycol or a combination of two or more thereof, in some embodiments, polyethylene glycol 200, polyethylene glycol 300, polyethylene glycol 400, or a combination of two or more thereof. In some embodiments, the at least one vehicle according to e) is polyethylene glycol 300.

In some embodiments of the compositions described above the polysorbate according to b) is polyoxyethylene(20) sorbitan monooleate, the C₂-C₄ alcohol according to c) is absolute ethanol, the organic acid according to d) is anhydrous citric acid, and the polyethylene glycol according to e) is polyethylene glycol 300. In some embodiments, the cabazitaxel is cabazitaxel monohydrate.

In some embodiments, the compositions described above include:
a) from 0.9 to 1.1 weight-% of the cabazitaxel monohydrate or anhydrous cabazitaxel;
b) from 23 to 27 weight-% of the at least one surfactant, wherein is polyoxyethylene(20) sorbitan monooleate;
c) from 17 to 21 weight-% of the at least one C₂-C₄ alcohol, wherein the at least one C₂-C₄ alcohol is absolute ethanol;
d) from 0.35 to 0.55 weight-% of the at least one organic acid, wherein the at least one organic acid is anhydrous citric acid;
e) from 50 to 57 weight-% of the vehicle, wherein the vehicle is polyethylene glycol 300;
f) from 0 to 1 weight-% of water;
in each case based on the total weight of the composition, wherein at least 99 weight-% of the composition includes the components a), b), c), d), and e).

In some embodiments, the compositions described above include:
a) from 0.92 to 1.01 weight-% of cabazitaxel monohydrate or anhydrous cabazitaxel, in some embodiments, cabazitaxel monohydrate;
b) from 23 to 27 weight-% of the at least one surfactant, wherein the at least one surfactant is polyoxyethylene (20) sorbitan monooleate;
c) from 17 to 21 weight-% of the at least one C₂-C₄ alcohol, wherein the at least one C₂-C₄ alcohol is absolute ethanol;
d) from 0.4 to 0.5 weight-% of the at least one organic acid, wherein the at least one organic acid is anhydrous citric;
e) from 50 to 57 weight-% of the at least one vehicle, wherein the at least one vehicle is polyethylene glycol 300;
f) from 0 to 1 weight-% of water;
in each case based on the total weight of the composition, wherein at least 99 weight-% of the composition includes the components a), b), c), d), and e).

In some embodiments, the pharmaceutical compositions include:
a) from 0.5 to 1.5 weight-% of cabazitaxel;
b) from 20 to 40 weight-% of a polysorbate;
c) from 10 to 23 weight-% of a C₂-C₄ alcohol;
d) from 0.01 to 1 weight-% of an organic acid;
e) from 40 to 60 weight-% of a polyethylene glycol;
f) from 0 to 5 weight-% of water;
in each case based on the total weight of the composition.

In some embodiments, the compositions described above include:
a) from 0.7 to 1.3 weight-% of cabazitaxel;
b) from 20 to 30 weight-% of a polysorbate;
c) from 15 to 22 weight-% of a C₂-C₄ alcohol;
d) from 0.1 to 0.8 weight-% of an organic acid;
e) from 45 to 60 weight-% of a polyethylene glycol;
f) from 0 to 3 weight-% of water;
in each case based on the total weight of the composition.

In some embodiments, the compositions described above include:
a) from 0.92 to 1.01 weight-% of cabazitaxel;
b) from 23 to 27 weight-% of the polysorbate;
c) from 17 to 21 weight-% of the C₂-C₄ alcohol;
d) from 0.3 to 0.6 weight-% of the organic acid;
e) from 50 to 57 weight-% of the polyethylene glycol;
f) from 0 to 1 weight-% of water;
in each case based on the total weight of the composition.

In some embodiments of the compositions described above the polysorbate is polyoxyethylene (20) sorbitan monooleate, the C₂-C₄ alcohol is ethanol; the organic acid is citric acid; and the polyethylene glycol is polyethylene glycol 300. In some embodiments, the ethanol is absolute ethanol. In some embodiments, the citric acid is anhydrous citric acid.

In some embodiments of the compositions described above the cabazitaxel is cabazitaxel monohydrate.

In some embodiments of the compositions described above at least 95 weight-%, in some embodiments, at least 97 weight-%, in some embodiments, at least 99 weight-% of the composition includes the components a), b), c), d), and e).

In some embodiments, the compositions described above are liquids at a temperature in the range of from 24 to 26° C. and a pressure in the range of from 0.95 to 1.05 bar.

In some embodiments, the compositions described above have a concentration of cabazitaxel in the range of from 9 to 11 mg/ml.

In some embodiments described above, the compositions have a pH in the range of from 2 to 4, wherein the pH relates to the pH of the composition diluted with deionized water at a ratio of composition relative to water of 1:9, the pH value being determined with a pH meter comprising a pH sensitive glass electrode at a temperature of the diluted composition in the range of from 15 to 30° C. In some embodiments, the pH is in the range of from 2.8 to 3.5. In some embodiments, the temperature of the diluted composition is in the range from 20 to 25° C.

In some embodiments, the compositions described above are contained in a vial. In some embodiments, the vial includes from 42 to 48 mg of the cabazitaxel. In some embodiments, the vial includes from 57 to 63 mg of the cabazitaxel. In some embodiments, the vial is an air-tight vial. In some embodiments, the air-tight vial includes an air-tight stopper.

In some embodiments, the compositions described above include less than 5% dissolved oxygen, in some embodiments, less than 4% dissolved oxygen, in some embodiments, less than 3% dissolved oxygen. In some embodiments, the compositions include less than 3% dissolved oxygen.

In some embodiments of the compositions described above, the compositions include less than 3% oxygen in the headspace, in some embodiments, less than 2% oxygen in the headspace, in some embodiments, less than 1% oxygen in the headspace.

In some embodiments, the pharmaceutical compositions include:
a) from 0.92 to 1.01 weight-% of cabazitaxel monohydrate or anhydrous cabazitaxel, in some embodiments, cabazitaxel monohydrate;
b) from 23 to 27 weight-% of the at least one surfactant, wherein the at least one surfactant is polyoxyethylene (20) sorbitan monooleate;
c) from 17 to 21 weight-% of the at least one C₂-C₄ alcohol, wherein the at least one C₂-C₄ alcohol is absolute ethanol;
d) from 0.4 to 0.5 weight-% of the at least one organic acid, wherein the at least one organic acid is anhydrous citric;
e) from 50 to 57 weight-% of the at least one vehicle, wherein the at least one vehicle is polyethylene glycol 300;
f) from 0 to 1 weight-% of water;
in each case based on the total weight of the composition, wherein at least 99 weight-% of the composition includes components a), b), c), d), and e), wherein the composition is contained in an air-tight vial including an air-tight stopper, wherein the headspace of the vial includes less than 1% oxygen. In some embodiments, the composition includes less than 5% dissolved oxygen, in some embodiments, less than 4% dissolved oxygen, in some embodiments, less than 3% dissolved oxygen.

In some embodiments, one-vial pharmaceutical compositions include:
a) from 9.5 to 10.5 mg/ml of cabazitaxel;
b) from 238 to 280 mg/ml of polyoxyethylene (20) sorbitan monooleate;
c) from 176 to 218 mg/ml of absolute ethanol;
d) from 4 to 5 mg/ml of anhydrous citric acid;
e) from 520 to 590 mg/ml polyethylene glycol 300;
f) from 0 to 1 mg/ml of water,
wherein the one-vial pharmaceutical composition has a total volume of at least 4.5 ml.

In some embodiments, the one-vial pharmaceutical compositions described herein include an air-tight vial including an air-tight stopper.

In some embodiments, the one-vial pharmaceutical compositions described above include less than 3% oxygen in the headspace of the vial, in some embodiments less than 2% oxygen in the headspace, in some embodiments less than 1% oxygen in the headspace.

In some embodiments, the one-vial pharmaceutical compositions described herein include less than 5% dissolved oxygen, in some embodiments less than 4% dissolved oxygen, in some embodiments less than 3% dissolved oxygen.

In some embodiments of the one-vial pharmaceutical compositions described herein the total volume is 4.6 to 5.4 ml. In some embodiments, the total volume is 6.2 to 7.0 ml.

In some embodiments, a process for preparing at least some of the pharmaceutical compositions described above include mixing cabazitaxel or the pharmaceutically acceptable salt, solvate, or hydrate thereof, in some embodiments, cabazitaxel monohydrate or anhydrous cabazitaxel, the at least one surfactant, the at least one C₂-C₄ alcohol, the at least one organic acid, and the at least one vehicle, obtaining the composition, wherein the cabazitaxel or the pharmaceutically acceptable salt, solvate, or hydrate thereof, in some embodiments, cabazitaxel monohydrate or anhydrous cabazitaxel, the at least one surfactant, the at least one C₂-C₄ alcohol, the at least one organic acid, and the at least one vehicle are employed in amounts such that the composition includes, based on the total weight of the composition,
a) from 0.5 to 1.5 weight-%, in some embodiments from 0.7 to 1.3 weight-%, in some embodiments from 0.92 to 1.01 weight-% of the cabazitaxel or the pharmaceutically acceptable salt, solvate, or hydrate thereof, in some embodiments cabazitaxel monohydrate or anhydrous cabazitaxel;
b) from 20 to 40 weight-%, in some embodiments from 20 to 30 weight-%, in some embodiments y from 23 to 27 weight-% of the at least one surfactant;
c) from 10 to 23 weight-%, in some embodiments from 15 to 22 weight-%, in some embodiments from 17 to 22 weight-% of the at least one C₂-C₄ alcohol;
d) from 0.01 to 1 weight-%, in some embodiments from 0.1 to 0.8 weight-%, in some embodiments from 0.3 to 0.6 weight-% of the at least one organic acid;
e) from 40 to 60 weight-%, in some embodiments from 45 to 60 weight-%, in some embodiments from 50 to 57 weight-% of the at least one vehicle is a polyethylene glycol or a combination of two or more thereof;
f) from 0 to 5 weight-%, in some embodiments 0 to 3 weight-%, more preferably from 0 to 1 weight-% of water.

In some embodiments, in the processes described above the at least one surfactant according to b) includes polyoxyethylene (20) sorbitan monooleate, wherein the at least one C₂-C₄ alcohol according to c) includes absolute ethanol, wherein the at least one organic acid according to d) includes anhydrous citric acid, and wherein the at least one vehicle according to e) includes polyethylene glycol 300.

In some embodiments, the processes described above include:
(i) dissolving the at least one organic acid in the at least one C₂-C₄ alcohol, obtaining a mixture I;
(ii) dissolving the cabazitaxel or the pharmaceutically acceptable salt, solvate, or hydrate thereof, in some embodiments cabazitaxel monohydrate or anhydrous cabazitaxel, in the mixture I, obtaining a mixture II;
(iii) dissolving the at least one surfactant in the mixture II, obtaining a mixture III;
(iv) adding the at least one vehicle to the mixture III, obtaining a mixture IV;
(v) mixing the mixture IV, obtaining the composition.

In some embodiments, the processes described above further include purging the solution with nitrogen after step (v).

In some embodiments, in the processes described above the compositions include less than 5% dissolved oxygen, in some embodiments less than 4% dissolved oxygen, n some embodiments less than 3% dissolved oxygen.

In some embodiments, vials including the pharmaceutical compositions described above include an inert gas, in some embodiments, nitrogen.

In some embodiments, the vials described above include an air-tight stopper wherein the headspace of the vial includes less than 3% oxygen, in some embodiments less than 1% oxygen.

In some embodiments, the vials, the one-vial pharmaceutical compositions, or the compositions described above include from 20 to 90 mg, in some embodiments from 30 to 75 mg, in some embodiments from 35 to 65 mg of the cabazitaxel or the pharmaceutically acceptable salt, solvate, or hydrate thereof, in some embodiments cabazitaxel monohydrate or anhydrous cabazitaxel, and in some embodiments, from 35 to 45 mg of the cabazitaxel or the pharmaceutically acceptable salt, solvate, or hydrate thereof, in some embodiments cabazitaxel monohydrate or anhydrous cabazitaxel.

In some embodiments, the vials, one-vial pharmaceutical compositions, or the compositions described herein include from 42 to 48 mg of the cabazitaxel or the pharmaceutically acceptable salt, solvate, or hydrate thereof, in some embodiments, cabazitaxel monohydrate, in some embodiments, from 55 to 65 mg of the cabazitaxel or the pharmaceutically acceptable salt, solvate, or hydrate thereof, in some embodiments, cabazitaxel monohydrate or anhydrous cabazitaxel, and in some embodiments, from 57 to 63 mg of the cabazitaxel or the pharmaceutically acceptable salt, solvate, or hydrate thereof, in some embodiments cabazitaxel monohydrate.

In some embodiments, parenteral formulations include the pharmaceutical compositions described above dissolved in a parenteral fluid. In some embodiments, the parenteral fluid is a saline solution, a glucose solution, a dextran solution, or a mixture of two or more thereof.

In some embodiments, the parenteral fluid is a saline solution including from 0.5 to 1.5 weight-%, in some embodiments from 0.75 to 1.0 weight-% of sodium chloride, in some embodiments 0.9 weight-% of sodium chloride.

In some embodiments, the parenteral fluid is a glucose solution including from 3 to 7 weight-%, in some embodiments from 4 to 6 weight-% of glucose, in some embodiments 5 weight-% of glucose.

In some embodiments, the parenteral formulation includes cabazitaxel monohydrate or anhydrous cabazitaxel, at a concentration of from 0.1 to 0.26 mg/ml.

In some embodiments, the parenteral formulations described herein are intravenous formulations.

In some embodiments, processes for preparing the parenteral formulations described above include:
A) providing the pharmaceutical compositions as described herein;
B) admixing the composition provided according to A) with a parenteral fluid in some embodiments being a saline solution, a glucose solution, a dextran solution, or a mixture of two or more thereof, wherein in some embodiments, the parenteral fluid is a saline solution including from 0.5 to 1.5 weight-%, in some embodiments from 0.75 to 1.0 weight-% of sodium chloride, in some embodiments 0.9 weight-% of sodium chloride, or a glucose solution including from 3 to 7 weight-%, in some embodiments from 4 to 6 weight-% of glucose, in some embodiments 5 weight-% of glucose;
including cabazitaxel at a concentration of from 0.1 to 0.26 mg/ml.

In some embodiments, the compositions, one-vial pharmaceutical compositions, or vials described herein can be used in a method of treating prostate cancer.

In some embodiments, the compositions, one-vial pharmaceutical compositions, or vials described herein are used in combination with prednisone or prednisolone.

In some embodiments, methods for the treatment of the human or animal body include administering a parenteral formulation as described herein. In some embodiments, the parenteral formulation is administered in combination with prednisone or prednisolone. In some embodiments, the parenteral formulations described herein are used in a method for the treatment of the human or animal body. In some embodiments, the parenteral formulations described herein in combination with prednisone or prednisolone for are used in a method for the treatment of the human or animal body.

In some embodiments, methods for the treatment of prostate cancer include administering a parenteral formulation as described herein to a patient in need thereof. In some embodiments, the methods further include administering the parenteral formulation in combination with prednisone or prednisolone.

In some embodiments, the parenteral formulations described herein in combination with prednisone or prednisolone are useful for methods for the treatment of prostate cancer.

In some embodiments, use of parenteral formulations include use for the preparation of a medicament for the treatment of prostate cancer. In some embodiments, use further includes use in combination with prednisone or prednisolone.

The pharmaceutical compositions are illustrated by the following examples.

### EXAMPLES

The following methods were used to determine the pH, the stability of compositions including cabazitaxel, the identity of cabazitaxel, the concentration of cabazitaxel, the clarity of the formulations described herein, the ethanol contents, the total impurities, and the critical micelle concentration of infusion solutions.

### Method 0

### Test Conditions

The cabazitaxel compositions were stored in glass vials at the temperature reported in Tables 1 and 2 or at a temperature and relative humidity (r.h.) as reported in Tables 3 to 8. The vials were stored either in upright or inverted position. In the inverted position the cabazitaxel composition is in contact with the stopper. The vials were stored for a determinate period of time as reported in the Tables 1 to 8. At the end of the each period, the identity of cabazitaxel, the concentration of cabazitaxel, the clarity of the solution, the pH, the ethanol concentration and the total impurities were measured according to Methods 1, 2, 4 and 5 as reported below.

### Method 1

### pH

1 ml of the composition is diluted with 9 ml of purified water and the pH of the resulting mixture was measured according to the European Pharmacopoeia 2.2.3 at a temperature in the range of from 20 to 25° C. using a potentiometric pH meter (e.g. pH-Meter 867 metrohm) calibrated using buffer solutions of known pH (e.g. pH 2.00, 4.01, 7.00 and 10.00).

### Method 2

### Identity, Assay, and Cabazitaxel Related Impurity Level

The identity, assay and cabazitaxel related impurity of the cabazitaxel compositions prepared in Examples 4 and 5 were determined using the following HPLC method.

### Preparation of Assay Standards:

Two standards (assay and assay standard agreement) are prepared from separate weighings to make standard 1a and standard 1b, each with a nominal cabazitaxel concentration of 0.50 mg/ml. To prepare each of these two standards, 25.0 mg cabazitaxel is weighed into a 50 ml amber glass volumetric flask and 20.0 ml acetonitrile is added. The solution is sonicated for approximately 5 minutes, until all if the cabazitaxel is dissolved. The solution is left to cool to room temperature. Deionized water is then added to a final volume of 50 ml. The 1a standard (assay) is injected five times, while the 1b standard (assay standard agreement) is injected once as part of the system suitability test.

### Preparation of Impurity Standard:

A further dilution to a nominal cabazitaxel concentration of 0.005 mg/ml cabazitaxel is prepared by diluting 0.5 ml of the 1a standard with a solution of 40:60 v/v acetonitrile/deionized water to a final volume of 50 ml in a 50 ml amber glass volumetric flask. This solution is injected six times as part of the system suitability test.

### Preparation of Test Samples:

0.5 ml of the cabazitaxel composition to be analyzed is diluted with 40:60 v/v acetonitrile/deionized water to a final volume of 10 ml.

The 1a standard is used for the identification of cabazitaxel in the compositions to be assayed. The retention time of the cabazitaxel peak in each assayed composition is compared to the retention time of cabazitaxel in the 1a standard. The cabazitaxel assay values for the composition are calculated by means of integration against external standard using the response factor obtained from assays standards 1a and 1b. The levels of unspecified unidentified impurities are calculated by means of integration against external standard using the response factor obtained from the impurity standard. Each formulation standard is injected singularly.

The HPLC method using the Agilent 1290 System (or alternatively Agilent 1260 system) is performed as follows:
column: Zorbax Eclipse Plus C18 RRHD, 1.8 μm, 50×2.1 mm ID
inline filter: 0.3 μm Inline Filter (for example Agilent 1290 Inline Filer Art. 5023-0271)
needle wash: 90:10 v/v methanol:deionized water
diluting solution: 40:60 v/v acetonitrile:deionized water
mobile phase: A) 25:75:0.1 v/v/v acetonitrile:deionized water:formic acid
   B) 100:0.1 v/v acetonitrile:formic acid (100:0.1)
gradient:

| time (minutes) | % B | % A |
|---|---|---|
| 0.0 | 0 | 100 |
| 2.0 | 0 | 100 |
| 5.5 | 28 | 72 |
| 10.0 | 46 | 54 |
| 10.1 | 100 | 0 |
| 13.0 | 100 | 0 |
| 13.1 | 0 | 100 |
| 15.0 | 0 | 100 |

Post-time: approximately 7 minutes (if using Agilent 1260 system or equivalent system)
injection volume: 5 μl
detector: UV, 230 nm, bandwidth 4 nm (DAD), reference 440 nm, bandwidth: 100 nm
flow rate: 0.4 ml/min
column temperature: 45° C.
run time: 15 minutes
autosampler temperature: room temperature

### Method 3

### Alternative HPLC Method for Identity, Assay, and Cabazitaxel Related Impurity Level

The identity, assay and cabazitaxel related impurity of the cabazitaxel compositions prepared in Examples 1, 2 and 3 were determined using the following HPLC method.

### Preparation of Assay Standards:

Two standards (assay and assay standard agreement) are prepared from separate weighings to make standard 1a and standard 1b, each with a nominal cabazitaxel concentration 1.0 mg/ml. To prepare each of these two standards, 25.0 mg cabazitaxel is weighed into a 25 ml volumetric flask and 20.0 ml of a solution of 40:60 v/v acetonitrile/deionized water is added. The solution is sonicated for approximately 10 minutes and allowed to cool to room temperature. A solution of 40:60 v/v acetonitrile/deionized water is then added to a final volume of 25 ml. The 1a standard (Assay) is injected five times, while the 1b standard (assay standard agreement) is injected once as part of the system suitability test.

### Preparation of Impurity Standard:

A further dilution to nominal cabazitaxel concentration of 0.005 mg/ml cabazitaxel is prepared by diluting 0.5 ml of the 1a standard in a 100 ml volumetric flask with a solution of 40:60 v/v acetonitrile/deionized water to a final volume of 100 ml. This solution is injected six times as part of the system suitability test.

### Preparation of Test Samples:

1.0 ml of the cabazitaxel composition to be analyzed is diluted with 40:60 v/v acetonitrile/deionized water to a final volume of 10 ml.

The HPLC method using a Agilent 1290 System, Agilent 1260 system, or an equivalent system is performed using the following conditions:
diluting solution: 40:60 v/v acetonitrile:deionized water
column: Zorbax Eclipse Plus C18 RRHD, 1.8 μm, 50×2.1 mm ID
inline filter: 2 mm×0.2 μm RRLC inline Filter
mobile phase: A) 40:60 v/v acetonitrile:deionized water
   B) acetonitrile
gradient:

| time (minutes) | % B | % A |
|---|---|---|
| 0.0 | 0 | 100 |
| 3.8 | 0 | 100 |
| 10.0 | 50 | 50 |
| 12.5 | 60 | 40 |
| 13.1 | 100 | 0 |
| 16.3 | 100 | 0 |
| 16.8 | 0 | 100 |
| 20.0 | 0 | 100 |

injection volume: 4 μl
detector: UV, 233 nm (DAD)
flow rate: 0.4 ml/min
column temperature: 40° C.
run time: 20 minutes
autosampler temperature: room temperature

### Method 4

### Clarity of a Solution

The clarity of a solution of the compositions prepared in Examples 4 and 5 was determined. The clarity of the solution is measured according to the European Pharmacopoeia 2.2.1 and USP 641 as follows at room temperature and atmospheric pressure.

Reference suspensions 1-IV (suspension ref. I is the clearest suspension and suspension ref. IV is the least clear suspension) are prepared according to the European Pharmacopoeia 2.2.1. The samples of cabazitaxel formulation to be tested are visually compared to the reference suspensions and their clarity was visually rated in relation to reference suspensions I to IV. Therefore, a sample with a clarity of less than I means that it is found to be clearer than the clearest reference suspension.

### Method 5

### EtOH

The content of ethanol in the formulations of Examples 4 and 5 was assessed by gas chromatograph (GC) equipped with a liquid autoinjector and a flame ionization detector (FID) using the following method.

### Preparation of Internal Standard:

An internal standard solution is prepared by adding 5000.0 mg (6218.91 μl) 1-propanol (density=0.804 g/ml) into a 50 ml volumetric flask and diluting to a final volume of 50 ml with methanol.

### Preparation of Ethanol Standard:

An ethanol standard is prepared by adding 95.88 mg (121.37 μl) ethanol (density=0.790 g/ml) and 1.50 ml of the internal standard solution into a 50 ml volumetric flask and diluting to a final volume of 50 ml with methanol. Nominal concentrations: 1.9177 mg/ml ethanol and 3.0000 mg/ml 1-propanol.

### Preparation of Test Samples:

500.0 mg (±50 mg) of the composition to be analyzed is added into a 50 ml volumetric flask. 1.50 ml of the internal standard solution is added and methanol is added to a final volume of 50 ml.

The following conditions are used for the GC analysis:
column: 6% cyanopropylphenyl-94% dimethylpolysiloxane, L=30 m, ID=0.53 mm, film thickness=3.0 μm (for example DB 624 from J&W Scientific). Stationary phase USP G43
injection volume 1 μl
injector: split
injector type: Agilent 6890N: Split 1:100 (1 part column, 100 parts waste)
injector temperature: 250° C.
detector temperature: 300° C.
carrier gas: Helium
total flow: Agilent 6890N: ˜808 ml/min
column flow rate: 8.0 ml/min constant flow
oven temperature: 50° C. for 3.5 minutes
   50° C./min to 250° C.
   250° C. for 4 minutes
FID detector: air 400 ml/min
   hydrogen 30 ml/min

### Example 1

1 ml formulations were prepared including the following:

| | | Ethanol | Citric acid | | | |
|---|---|---|---|---|---|---|
| Formulation | Cabazitaxel | (absolute) | (anhydrous) | PS 80 | PEG 300 | Headspace |
| No. | (mg/ml) | (ml/ml) | (mg/ml) | (ml/ml) | (ml/ml) | Gas |
| 1A | 10 | 0.15 | 0.21 | 0.25 | ~0.60 | air |
| 1E1 | 10 | 0.30 | 0.21 | 0.25 | ~0.45 | air |
| 1E2 | 10 | 0.13 | 0.21 | 0.25 | ~0.62 | air |
| 1E3 | 10 | 0.75 | 0.21 | 0.25 | — | air |
| 1E4 | 10 | 0.20 | 0.21 | 0.25 | ~0.55 | air |
| 1C1 | 10 | 0.15 | 4.0 | 0.25 | ~0.60 | air |
| 1C2 | 10 | 0.15 | 0.10 | 0.25 | ~0.60 | air |
| 1C3 | 10 | 0.15 | 0.30 | 0.25 | ~0.60 | air |
| 1C4 | 10 | 0.15 | — | 0.25 | ~0.60 | air |
| 1C5 | 10 | 0.20 | 3.0 | 0.25 | ~0.55 | air |
| 1C6 | 10 | 0.20 | 5.0 | 0.25 | ~0.55 | air |
| 1N | 10 | 0.15 | 0.21 | 0.25 | ~0.60 | N₂ |

All formulations were prepared with cabazitaxel monohydrate.

After preparation, the percentage of cabazitaxel and impurities were assayed by HPLC using Method 3. The relative pH reported was determined with an ethanol electrode (Et-OH Trode from Metrohm—6.0269.100). The threshold applied for an impurity was detection above 0.05%. Accordingly, the total impurities reported below includes all impurities detected above 0.05%.

0 Time Point

| Formula- | Cabazitaxel | Total impurities | Relative |
|---|---|---|---|
| tion No. | (%) | (%) | pH |
| 1A | 87.04 | 0.81 | 7.8 |
| 1E1 | 89.55 | 0.72 | 7.7 |
| 1E2 | 84.88 | 0.46 | 7.8 |
| 1E3 | 94.35 | 0.55 | 6.2 |
| 1E4 | 84.02 | 0.65 | 7.6 |
| 1C1 | 89.44 | 0.23 | 5.3 |
| 1C2 | 87.45 | 0.26 | 8.1 |
| 1C3 | 89.67 | 0.26 | 7.7 |
| 1C4 | 88.74 | 0.32 | 8.7 |
| 1C5 | 95.22 | 0.37 | 5.5 |
| 1C6 | 94.42 | 0.42 | 4.8 |
| 1N | 89.62 | 0.27 | 7.8 |

2 Months at 40° C.

| Formula- | Cabazitaxel | Total impurities | Relative |
|---|---|---|---|
| tion No. | (%) | (%) | pH |
| 1A | 91.73 | 5.46 | 7.8 |
| 1E1 | 94.53 | 4.13 | 7.6 |
| 1E2 | 92.37 | 4.98 | 7.8 |
| 1E3 | 97.17 | 0.78 | 6.4 |
| 1E4 | 101.84 | 4.17 | 7.6 |
| 1C1 | 94.29 | 1.32 | 5.5 |
| 1C2 | 92.83 | 11.11 | 8.0 |
| 1C3 | 96.16 | 4.69 | 7.7 |
| 1C4 | 70.58 | 31.41 | 8.7 |
| 1C5 | 96.77 | 3.05 | 5.5 |
| 1C6 | 93.52 | 0.94 | 5.1 |
| 1N | 88.54 | 5.57 | 7.9 |

Phase separation in the formulations became visible first at 1-8° C., followed by 25° C. and at last at 40° C. The 1E2 formulation with 13% (v/v) ethanol (approximately 103 mg/ml; approximately 10 weight-%) showed phase separation at all three temperatures by 2 months indicating that 13% v/v ethanol is below the level required to keep the cabazitaxel composition in one phase. No phase separation for formulations comprising at least 20% (v/v) ethanol (approximately 158 mg/ml; approximately 15 weight-%) was observed. Although a high level of ethanol protects the formulation from degradation, a lower level is required for safety purposes.

The introduction of nitrogen in the headspace in sample formulation 1N was not efficient and therefore any advantage of nitrogen in the headspace could not be disregarded based on this sample.

### Example 2

1 ml formulations were prepared including the following:

| | | Ethanol | Citric acid | | |
|---|---|---|---|---|---|
| Formula- | Cabazitaxel | (absolute) | (anhydrous) | PS 80 | PEG 300 |
| tion No. | (mg/ml) | (ml/ml) | (mg/ml) | (ml/ml) | (ml/ml) |
| 1C6 | 10 | 0.30 | 5.0 | 0.25 | ~0.45 |
| 1C7 | 10 | 0.25 | 4.5 | 0.25 | ~0.5 |
| 1C8 | 10 | 0.25 | 4.0 | 0.25 | ~0.5 |
| 1E4 | 10 | 0.25 | 5.0 | 0.25 | ~0.5 |
| 1E5 | 10 | 0.23 | 4.5 | 0.25 | ~0.52 |
| 1E6 | 10 | 0.20 | 4.5 | 0.25 | ~0.55 |
| 1E7 | 10 | 0.28 | 4.5 | 0.25 | ~0.47 |
| 1E8 | 10 | 0.30 | 4.5 | 0.25 | ~0.45 |
| 1E9 | 10 | 0.20 | 5.0 | 0.25 | ~0.55 |
| 2C7 | 10 | 0.25 | 4.5 | 0.25 | ~0.5 |

For all the formulations the stopper was a West chlorobutyl rubber stopper with fluorotec coating. All formulations except 2C7 were prepared from cabazitaxel ethyl acetate. 2C7 was prepared with cabazitaxel monohydrate.

After preparation, the percentage of cabazitaxel and impurities were assayed by HPLC using Method 3. The relative pH reported was determined with an ethanol electrode (Et-OH Trode from Metrohm—6.0269.100). The threshold applied for an impurity was detection above 0.05%. Accordingly, the total impurities reported below include all impurities detected above 0.05%.

0 Time Point

| | | Total | |
|---|---|---|---|
| Formulation | Cabazitaxel | impurities | |
| No. | (%) | (%) | Relative pH |
| 1C6 | 102.9 | 0.10 | 5.2 |
| 1C7 | 94.9 | 0.05 | 5.2 |
| 1C8 | 100.8 | 0.00 | 5.0 |
| 1E4 | 99.9 | 0.00 | 5.4 |
| 1E5 | 99.7 | 0.00 | 5.5 |
| 1E6 | 97.7 | 0.00 | 5.4 |
| 1E7 | 102.7 | 0.00 | 5.4 |
| 1E8 | 101.9 | 0.00 | 5.3 |
| 1E9 | 95.4 | 0.00 | 5.3 |
| 2C7 | 108.0 | 0.05 | 5.3 |

3 Months at 40° C.

| | | Total | |
|---|---|---|---|
| Formulation | Cabazitaxel | impurities | |
| No. | (%) | (%) | Relative pH |
| 1C6 | 102.3 | 0.25 | 5.1 |
| 1C7 | 105.1 | 0.20 | 5.1 |
| 1C8 | 103.8 | 0.19 | 5.3 |
| 1E4 | 103.6 | 0.25 | 5.3 |
| 1E5 | 103.5 | 0.20 | 5.5 |
| 1E6 | 102.3 | 0.20 | 5.4 |
| 1E7 | 104.4 | 0.25 | 5.3 |
| 1E8 | 104.2 | 0.28 | 5.1 |
| 1E9 | 102.1 | 0.25 | 5.2 |
| 2C7 | 110.7 | 0.20 | 5.2 |

No phase separation was observed in any formulation. Higher levels of degradation were observed with increasing ethanol concentration. In the 1E6, 1E5, 1C7, 1E7, 1E8 formulations, with ethanol levels of 0.20, 0.23, 0.25, 0.28, 0.30 ml/ml, respectively, the level of ethanol was the only difference between the formulations. It can be seen that 1E8, with the highest level of ethanol, demonstrated the highest level of degradation.

### Example 3

This example was designed to evaluate variation of the ingredients: 4.2 to 4.9 mg/ml citric acid (anhydrous) (approximately 0.40 weight-% to 0.47 weight-%), 23 to 27% (v/v) ethanol (absolute) (approximately 181 mg/ml to 213 mg/ml; approximately 17.5 weight-% to 20.5 weight-%). In addition, the impact of oxidized polysorbate 80 was evaluated (formulation 6C70). The oxidized polysorbate 80 was prepared by exposure to air for 3 months by a hole in the container. The formulations were prepared and filled into vials with a total capacity of 10 ml (tubular type 1 6 ml glass vials). The impact of light on the formulations was also evaluated by covering vials with the same formulation at 6C7 with foil (samples designated 6C7L1). All formulations were prepared with cabazitaxel monohydrate. All formulations except 6C7A were prepared by dissolving cabazitaxel in ethanol prior to addition of the citric acid. For 6C7A, citric acid was dissolved in ethanol prior to the addition of cabazitaxel. The stopper used was the West chlorobutyl rubber stopper with fluorotec coating.

1 ml formulations were prepared including the following:

| Formu- | | Ethanol | Citric acid | | |
|---|---|---|---|---|---|
| lation | Cabazitaxel | (absolute) | (anhydrous) | PS 80 | |
| No. | (mg/ml) | (ml/ml) | (mg/ml) | (g/ml) | PEG 300 |
| 2C7 | 10 | 0.25 | 4.5 | 0.26 | To 1 ml |
| 2C7E1 | 10 | 0.23 | 4.5 | 0.26 | To 1 ml |
| 2C7E2 | 10 | 0.27 | 4.5 | 0.26 | To 1 ml |
| 2C7C1 | 10 | 0.25 | 4.2 | 0.26 | To 1 ml |
| 2C7C2 | 10 | 0.25 | 4.9 | 0.26 | To 1 ml |
| 2C7P1 | 10 | 0.25 | 4.5 | 0.24 | To 1 ml |
| 2C7P2 | 10 | 0.25 | 4.5 | 0.28 | To 1 ml |
| 6C7P3 | 10 | 0.25 | 4.5 | 0.26 | To 1 ml |
| 6C7 | 10 | 0.25 | 4.5 | 0.26 | To 1 ml |
| 6C7L1 | 10 | 0.25 | 4.5 | 0.26 | To 1 ml |
| 6C7O | 10 | 0.25 | 4.5 | 0.26 | To 1 ml |
| | | | | (oxidized) | |
| 6C7A | 10 | 0.25 | 4.5 | 0.26 | To 1 ml |

After preparation, the percentage of cabazitaxel and impurities were assayed by HPLC using Method 3. The relative pH reported was determined with an ethanol electrode (Et-OH Trode from Metrohm—6.0269.100). The threshold applied for an impurity was detection above 0.05%. Accordingly, the total impurities reported below includes all impurities detected above 0.05%.

0 Time Point

| | | Total | |
|---|---|---|---|
| Formulation | Cabazitaxel | impurities | |
| No. | (%) | (%) | Relative pH |
| 2C7 | 101.71 | 0.00 | 5.37 |
| 2C7E1 | 101.40 | 0.00 | 5.34 |
| 2C7E2 | 103.06 | 0.00 | 5.34 |
| 2C7C1 | 100.19 | 0.00 | 5.16 |
| 2C7C2 | 104.07 | 0.00 | 5.28 |
| 2C7P1 | 102.04 | 0.00 | 5.23 |
| 2C7P2 | 105.57 | 0.06 | 5.25 |
| 6C7P3 | 101.63 | 0.00 | 5.54 |
| 6C7 | 103.02 | 0.00 | 5.15 |
| 6C7L1 | 103.02 | 0.00 | 5.15 |
| 6C7O | 99.04 | 0.08 | 5.40 |
| 6C7A | 103.34 | 0.00 | 5.36 |

1 Month at 40° C.

| | | Cabazitaxel | | |
|---|---|---|---|---|
| | | amount | | |
| | | with | Total | |
| Formulation | Cabazitaxel | respect to 0 | impurities | |
| No. | (%) | Time point | (%) | Relative pH |
| 2C7 | 102.31 | 100.59 | 0.00 | 5.34 |
| 2C7E1 | 102.38 | 100.97 | 0.05 | 5.28 |
| 2C7E2 | 102.41 | 99.37 | 0.05 | 5.23 |
| 2C7C1 | 102.50 | 102.31 | 0.00 | 5.36 |
| 2C7C2 | 104.08 | 100.01 | 0.06 | 5.29 |
| 2C7P1 | 100.95 | 98.93 | 0.05 | 5.24 |
| 2C7P2 | 105.36 | 99.80 | 0.05 | 5.35 |
| 6C7P3 | 101.21 | 99.59 | 0.05 | 5.52 |
| 6C7 | 102.45 | 99.45 | 0.11 | 5.16 |
| 6C7L1 | 102.24 | 99.24 | 0.11 | 5.33 |
| 6C7O | 100.66 | 101.64 | 0.21 | 5.38 |
| 6C7A | — | | 0.12 | 5.35 |

Higher impurities levels were observed in the formulation prepared with oxidized polysorbate 80. It was, therefore, decided to introduce nitrogen during the manufacturing process.

### Example 4

### Preparation of a 10 mg/ml Cabazitaxel Composition

Three batches of the cabazitaxel composition, as detailed in Table 1, were prepared using the following preparation method:

TABLE 1

| | Batch 1 | Batch 2 | Batch 3 |
|---|---|---|---|
| cabazitaxel | 0.479 kg | 0.481 kg | 0.480 kg |
| ethanol | 8.195 kg | 8.195 kg (portion 1) | 8.180 kg (portion 1) |
| (absolute) | (portion 1) | | |
| | 1.130 kg | 1.130 kg (portion 2) | 1.130 kg (portion 2) |
| | (portion 2) | | |
| citric acid | 0.212 kg | 0.212 kg | 0.212 kg |
| (anhydrous) | | | |
| polysorbate 80 | 12.181 kg | 12.202 kg | 12.209 kg |
| (super refined | | | |
| Tween 80-LQ- | | | |
| (MH)) | | | |
| polyethylene | 26.403 kg | 26.394 kg | 26.365 kg |
| glycol 300 | | | |

The formulation was compounded in a mixing vessel under a nitrogen atmosphere. Before addition of any component, the mixing vessel was flushing with nitrogen for at least 20 minutes. (The flushing times reported have been rounded to the nearest minute).

| | Batch 1 | Batch 2 | Batch 3 |
|---|---|---|---|
| Nitrogen flushing time | 22 minutes | 21 minutes | 31 minutes |
| (800 l/h) | | | |

The first portion of ethanol (absolute) was added into the mixing vessel followed by the addition of citric acid (anhydrous). The resulting mixture was stirred for at least 15 minutes. (All stirring times reported have been rounded to the nearest minute.) After stirring, the citric acid was completely dissolved.

| | Batch 1 | Batch 2 | Batch 3 |
|---|---|---|---|
| Stirring time | 17 minutes | 16 minutes | 16 minutes |

Cabazitaxel was then added stepwise with stirring. The stepwise addition of cabazitaxel was carried out such that each addition was dissolved before more was added into the mixing vessel. The second portion of ethanol (absolute) was used to wash the remaining cabazitaxel from the equipment used for its addition into the mixing vessel. After addition of the second portion of ethanol, stirring of the mixture was continued for at least 30 minutes. After stirring, the cabazitaxel was completely dissolved.

| | Batch 1 | Batch 2 | Batch 3 |
|---|---|---|---|
| Stirring time for total | 37 minutes | 32 minutes | 37 minutes |
| cabazitaxel addition | | | |

Polysorbate 80 (super refined Tween 80-LQ-(MH)) was then added and the mixture was stirred for at least 45 minutes.

| | Batch 1 | Batch 2 | Batch 3 |
|---|---|---|---|
| Stirring time | 51 minutes | 49 minutes | 48 minutes |

Polyetheylene glycol 300 was then was then added and the mixture was stirred for at least 45 minutes.

| | Batch 1 | Batch 2 | Batch 3 |
|---|---|---|---|
| Stirring time | 48 minutes | 47 minutes | 48 minutes |

The final mixture was then purged with nitrogen, by bubbling nitrogen through the mixture, for at least 15 minutes.

| | Batch 1 | Batch 2 | Batch 3 |
|---|---|---|---|
| Nitrogen flushing time | 16 minutes | 16 minutes | 22 minutes |
| (800 l/h) | | | |

The pH, density and dissolved air of the final formulations were determined.

| | Batch 1 | Batch 2 | Batch 3 |
|---|---|---|---|
| pH | (not available) | 3.223 (25.3° C.) | 3.267 (23.9° C.) |
| density | 1.038 g/cm³ | 1.038 g/cm³ | 1.039 g/cm³ |
| | (20.0° C.) | (20.0° C.) | (20.0° C.) |
| Dissolved Air (%) | 0.735 | 13.933 | 1.888 |
| in formulation | (0.154) | (2.926) | (0.397) |
| (estimated | | | |
| dissolved | | | |
| oxygen %) | | | |

The pH was measured by diluting 1 ml of the mixture with deionized water up to final volume of 10 ml. Density was measured with the DMA 4500M (Anton Paar) density meter. The concentration of air dissolved in the final formulations was measured using an InPro6000 (Mettler Toledo) amperometric oxygen sensor. The reported percentage of oxygen was calculated based on 21% of the measured dissolved air in the formulation.

### Example 5

### Preparation of Vials Comprising 45 mg or 60 mg of Cabazitaxel

The formulation prepared in Example 4 was dosed into clear glass vials with a total capacity of 10 ml. Filing volumes were targeted to 5.0 ml (from 4.65 to 5.35 ml) and 6.6 ml (from 6.20 to 7.00 ml). The filling volumes were controlled by weight for the 5.0 ml volume, the control was from 4.80 g to 5.52 g and for the 6.6 ml from 6.71 g to 7.23 g. After filling, the headspace of the vial was flushed with nitrogen prior to stoppering with a fluoropolymer-coated stopper (West Pharmaceutical Services Flurotec injection stopper).

The oxygen content of the vial headspace after preparation and before storage was determined using a Lighthouse Instruments Headspace Oxygen Analyzer Model FMS-760. This system uses a near-infrared laser that is tuned to match an internal absorption frequency of oxygen that is passed through the containing in the headspace above the solution. Standards were measured with the oxygen percentages of 0, 1.0, 2.0, 4.0, 8.0 and 20.0. Five vials from the following preparations were assessed in triplicate: Batch 1, 45 mg preparation (B1-45); Batch 1, 60 mg preparation (B1-60); Batch 2, 45 mg preparation (B1-45); and Batch 3, 45 mg preparation (B3-45).

T = 0

| Standard Oxygen (%) | Oxygen (%) |
|---|---|
| 0.0 | 0.149 |
| 1.0 | 1.133 |
| 2.0 | 1.931 |
| 4.0 | 4.133 |
| 8.0 | 8.251 |
| 20.0 | 20.096 |

| Sample | vial no. | Oxygen (%) |
|---|---|---|
| B1-45 | 1 | 0.361, 0.282, 0.246 |
| | 2 | 0.707, 0.758, 0.736 |
| | 3 | 0.219, 0.205, 0.163 |
| | 4 | 0.187, 0.262, 0.213 |
| | 5 | 0.380, 0.408, 0.375 |
| B1-60 | 1 | 0.511, 0.574, 0.612 |
| | 2 | 0.692, 0.737, 0.686 |
| | 3 | 0.302, 0.158, 0.194 |
| | 4 | 0.450, 0.376, 0.210 |
| | 5 | 0.097, 0.040, 0.121 |
| B2-45 | 1 | 0.196, 0.231, 0.261 |
| | 2 | 0.325, 0.283. 0.168 |
| | 3 | 0.207, 0.202, 0.255 |
| | 4 | 0.151, 0.249, 0.237 |
| | 5 | 0.237, 0.242, 0.406 |
| B3-45 | 1 | 0.116, 0.190, 0.005 |
| | 2 | 0.304, 0.439, 0.392 |
| | 3 | 0.540, 0.652, 0.676 |
| | 4 | 0.549, 0.412, 0.527 |
| | 5 | 0.389, 0.428, 0.396 |

The oxygen in the headspace in the vials was below 1%.

### Example 6

### Stability Tests

The vials prepared in Example 5 were assessed according to the above-defined Methods 1, 2, 4 and 5 under the following conditions (vials stored both upright and inverted) at 2-8° C., 25° C. at 60% relative humidity, 30° C. at 75% relative humidity, and 40° C. at 75% relative humidity. Vials from the following batches were assessed: Batch 1, 45 mg preparation (B1-45); Batch 1, 60 mg preparation (B1-60); Batch 2, 45 mg preparation (B1-45); and Batch 3, 45 mg preparation (B3-45).

The results and the conditions are reported in the tables below.

TABLE 1
Stability test of compositions of Examples 4 & 5-Storage 2-8° C. (vial inverted)

| | | Tested Parameter | | | | | |
|---|---|---|---|---|---|---|---|
| | | | | Cabazitaxel | | | |
| | | | | identity | | | |
| | | | | retention | | | |
| | | | | time | Cabazitaxel | | |
| | | | | corresponds | 90.0-110.0% | Total | |
| Shelf life | | Clarity | pH | to reference | (9.00-11.0 | Impurity | ethanol |
| specification | | <ref. I | 3.1-3.7 | (±3%) | mg/ml) | ≦3.0% | 90.0-110.0% |
| B1-60 | 0 point | <ref. I | 3.3 | Yes | 102.2 | <0.03 | 96.4 |
| | 6 months | <ref. I | 3.2 | Yes | 100.2 | <0.03 | 95.7 |
| B1-45 | 0 point | <ref. I | 3.3 | Yes | 101.3 | <0.03 | 95.8 |
| | 6 months | <ref. I | 3.2 | Yes | 100.4 | <0.03 | 93.0 |
| B2-45 | 0 point | <ref. I | 3.3 | Yes | 102.4 | <0.03 | 95.5 |
| | 6 months | <ref. I | 3.3 | Yes | 100.8 | <0.03 | 95.7 |
| B3-45 | 0 point | <ref. I | 3.3 | Yes | 101.9 | <0.03 | 95.3 |
| | 6 months | <ref. I | 3.2 | Yes | 101.0 | <0.03 | 94.3 |

Results are given as a mean of 3 determinations. As evident from Table 1, the composition solution remained clear under storage condition, no degradation of cabazitaxel and formation of impurity was observed, the pH of the solution remained unchanged. All these results indicate that the composition was stable. The additional contact with the stopper (inverted vial) as well did not alter the stability of the solution.

TABLE 2
Stability test of compositions of Examples 4 & 5-Storage 2-8° C. (vial upright)

| | | Tested Parameter | | | | | |
|---|---|---|---|---|---|---|---|
| | | | | Cabazitaxel | | | |
| | | | | identity | | | |
| | | | | retention | | | |
| | | | | time | Cabazitaxel | | |
| | | | | corresponds | 90.0-110.0% | Total | |
| Shelf life | | Clarity | pH | to reference | (9.00-11.0 | Impurity | ethanol |
| specification | | <ref. I | 3.1-3.7 | (±3%) | mg/ml) | ≦3.0% | 90.0-110.0% |
| B1-60 | 0 point | <ref. I | 3.3 | Yes | 102.2 | <0.03 | 96.4 |
| | 6 months | <ref. I | 3.2 | Yes | 100.8 | <0.03 | 96.4 |
| B1-45 | 0 point | <ref. I | 3.3 | Yes | 101.3 | <0.03 | 95.8 |
| | 6 months | <ref. I | 3.2 | Yes | 100.1 | <0.03 | 95.4 |
| B2-45 | 0 point | <ref. I | 3.3 | Yes | 102.4 | <0.03 | 95.5 |
| | 6 months | <ref. I | 3.2 | Yes | 100.0 | <0.03 | 94.0 |
| B3-45 | 0 point | <ref. I | 3.3 | Yes | 101.9 | <0.03 | 95.3 |
| | 6 months | <ref. I | 3.2 | Yes | 100.8 | <0.03 | 93.7 |

Results are given as a mean of 3 determinations. As evident from Table 2, the composition solution remained clear under storage condition, no degradation of cabazitaxel and formation of impurity was observed, the pH of the solution remained unchanged. All these results indicate that the composition was stable.

TABLE 3
Stability test of compositions of Examples 4 & 5-25° C./60% r.h. (vial upright)

| | | Tested Parameter | | | | | |
|---|---|---|---|---|---|---|---|
| | | | | Cabazitaxel | | | |
| | | | | identity | | | |
| | | | | retention | | | |
| | | | | time | Cabazitaxel | | |
| | | | | corresponds | 90.0-110.0% | Total | |
| Shelf life | | Clarity | pH | to reference | (9.00-11.0 | Impurity | ethanol |
| specification | | <ref. I | 3.1-3.7 | (±3%) | mg/ml) | ≦3.0% | 90.0-110.0% |
| B1-60 | 0 point | <ref. I | 3.3 | Yes | 102.2 | <0.03 | 96.4 |
| | 3 months | <ref. I | 3.3 | Yes | 100.3 | <0.03 | 93.1 |
| | 6 months | <ref. I | 3.3 | Yes | 100.6 | <0.03 | 96.3 |
| B1-45 | 0 point | <ref. I | 3.3 | Yes | 101.3 | <0.03 | 95.8 |
| | 3 months | <ref. I | 3.3 | Yes | 100.2 | <0.03 | 92.9 |
| | 6 months | <ref. I | 3.2 | Yes | 100.6 | <0.03 | 95.9 |
| B2-45 | 0 point | <ref. I | 3.3 | Yes | 102.4 | <0.03 | 95.5 |
| | 3 months | <ref. I | 3.3 | Yes | 100.6 | <0.03 | 92.6 |
| | 6 months | <ref. I | 3.2 | Yes | 99.0 | <0.03 | 96.0 |
| B3-45 | 0 point | <ref. I | 3.3 | Yes | 101.9 | <0.03 | 95.3 |
| | 3 months | <ref. I | 3.3 | Yes | 100.4 | <0.03 | 92.7 |
| | 6 months | <ref. I | 3.2 | Yes | 100.6 | <0.03 | 95.9 |

Results are given as a mean of 3 determinations. As evident from Table 3, the composition solution remained clear under the storage condition, no degradation of cabazitaxel and formation of impurity was observed, the pH of the solution remained substantially unchanged. All these results indicate that the composition was stable.

TABLE 4
Stability test of compositions of Examples 4 & 5-25° C./60% r.h. (vial inverted)

| | | Tested Parameter | | | | | |
|---|---|---|---|---|---|---|---|
| | | | | Cabazitaxel | | | |
| | | | | identity | | | |
| | | | | retention | | | |
| | | | | time | Cabazitaxel | | |
| | | | | corresponds | 90.0-110.0% | Total | |
| Shelf life | | Clarity | pH | to reference | (9.00-11.0 | Impurity | ethanol |
| specification | | <ref. I | 3.1-3.7 | (±3%) | mg/ml) | ≦3.0% | 90.0-110.0% |
| B1-60 | 0 point | <ref. I | 3.3 | Yes | 102.2 | <0.03 | 96.4 |
| | 3 months | <ref. I | 3.3 | Yes | 100.4 | <0.03 | 93.5 |
| | 6 months | <ref. I | 3.3 | Yes | 101.6 | <0.03 | 96.2 |
| B1-45 | 0 point | <ref. I | 3.3 | Yes | 101.3 | <0.03 | 95.8 |
| | 3 months | <ref. I | 3.3 | Yes | 100.2 | <0.03 | 93.0 |
| | 6 months | <ref. I | 3.2 | Yes | 100.5 | <0.03 | 96.2 |
| B2-45 | 0 point | <ref. I | 3.3 | Yes | 102.4 | <0.03 | 95.5 |
| | 3 months | <ref. I | 3.3 | Yes | 100.8 | <0.03 | 93.3 |
| | 6 months | <ref. I | 3.3 | Yes | 101.2 | <0.03 | 95.7 |
| B3-45 | 0 point | <ref. I | 3.3 | Yes | 101.9 | <0.03 | 95.3 |
| | 3 months | <ref. I | 3.3 | Yes | 100.2 | <0.03 | 92.7 |
| | 6 months | <ref. I | 3.3 | Yes | 101.0 | <0.03 | 95.5 |

Results are given as a mean of 3 determinations. As evident from Table 4, the composition solution remained clear under storage condition, no degradation of cabazitaxel and formation of impurity was observed, the pH of the solution remained unchanged. All these results indicate that the composition was stable. The additional contact with the stopper (inverted vial) as well did not alter the stability of the solution.

TABLE 5
Stability test of compositions of Examples 4 & 5-30° C./75% r.h. (vial upright)

| | | Tested Parameter | | | | | |
|---|---|---|---|---|---|---|---|
| | | | | Cabazitaxel | | | |
| | | | | identity | | | |
| | | | | retention | | | |
| | | | | time | Cabazitaxel | | |
| | | | | corresponds | 90.0-110.0% | Total | |
| Shelf life | | Clarity | pH | to reference | (9.00-11.0 | Impurity | ethanol |
| specification | | <ref. I | 3.1-3.7 | (±3%) | mg/ml) | ≦3.0% | 90.0-110.0% |
| B1-60 | 0 point | <ref. I | 3.3 | Yes | 102.2 | <0.03 | 96.4 |
| | 3 months | <ref. I | 3.3 | Yes | 100.3 | <0.03 | 92.7 |
| | 6 months | <ref. I | 3.3 | Yes | 100.5 | <0.03 | 95.5 |
| B1-45 | 0 point | <ref. I | 3.3 | Yes | 101.3 | <0.03 | 95.8 |
| | 3 months | <ref. I | 3.3 | Yes | 100.3 | <0.03 | 93.8 |
| | 6 months | <ref. I | 3.3 | Yes | 100.1 | <0.03 | 96.4 |
| B2-45 | 0 point | <ref. I | 3.3 | Yes | 102.4 | <0.03 | 95.5 |
| | 3 months | <ref. I | 3.3 | Yes | 100.6 | <0.03 | 93.3 |
| | 6 months | <ref. I | 3.3 | Yes | 101.6 | <0.03 | 95.7 |
| B3-45 | 0 point | <ref. I | 3.3 | Yes | 101.9 | <0.03 | 95.3 |
| | 3 months | <ref. I | 3.3 | Yes | 100.3 | <0.03 | 93.5 |
| | 6 months | <ref. I | 3.3 | Yes | 100.7 | <0.03 | 95.9 |

Results are given as a mean of 3 determinations. As evident from Table 5, the composition solution remained clear under storage condition, no degradation of cabazitaxel and formation of impurity was observed, the pH of the solution remained unchanged. All these results indicate that the composition was stable.

TABLE 6
Stability test of compositions of Examples 4 & 5-30° C./75% r.h. (vial inverted)

| | | Tested Parameter | | | | | |
|---|---|---|---|---|---|---|---|
| | | | | Cabazitaxel | | | |
| | | | | identity | | | |
| | | | | retention | | | |
| | | | | time | Cabazitaxel | | |
| | | | | corresponds | 90.0-110.0% | Total | |
| Shelf life | | Clarity | pH | to reference | (9.00-11.0 | Impurity | ethanol |
| specification | | <ref. I | 3.1-3.7 | (±3%) | mg/ml) | ≦3.0% | 90.0-110.0% |
| B1-60 | 0 point | <ref. I | 3.3 | Yes | 102.2 | <0.03 | 96.4 |
| | 3 months | <ref. I | 3.3 | Yes | 101.0 | <0.03 | 94.0 |
| | 6 months | <ref. I | 3.3 | Yes | 99.8 | <0.03 | 95.3 |
| B1-45 | 0 point | <ref. I | 3.3 | Yes | 101.3 | <0.03 | 95.8 |
| | 3 months | <ref. I | 3.3 | Yes | 100.4 | <0.03 | 93.7 |
| | 6 months | <ref. I | 3.3 | Yes | 99.9 | <0.03 | 97.2 |
| B2-45 | 0 point | <ref. I | 3.3 | Yes | 102.4 | <0.03 | 95.5 |
| | 3 months | <ref. I | 3.3 | Yes | 100.9 | <0.03 | 92.6 |
| | 6 months | <ref. I | 3.3 | Yes | 100.4 | <0.03 | 95.4 |
| B3-45 | 0 point | <ref. I | 3.3 | Yes | 101.9 | <0.03 | 95.3 |
| | 3 months | <ref. I | 3.3 | Yes | 100.5 | <0.03 | 92.6 |
| | 6 months | <ref. I | 3.3 | Yes | 99.9 | <0.03 | 95.1 |

Results are given as a mean of 3 determinations. As evident from Table 6, the composition solution remained clear under storage condition, no degradation of cabazitaxel and formation of impurity was observed, the pH of the solution remained unchanged. All these results indicate that the composition was stable. The additional contact with the stopper (inverted vial) as well did not alter the stability of the solution.

TABLE 7
Stability test of compositions of Examples 4 & 5-Storage 40° C./75% r.h. (vial inverted)

| | | Tested Parameter | | | | | |
|---|---|---|---|---|---|---|---|
| | | | | Cabazitaxel | | | |
| | | | | identity | | | |
| | | | | retention | | | |
| | | | | time | Cabazitaxel | | |
| | | | pH | corresponds | 90.0-110.0% | Total | ethanol |
| Shelf life | | Clarity | 3.1- | to reference | (9.00-11.0 | Impurity | 90.0- |
| specification | | <ref. I | 3.7 | (±3%) | mg/ml) | ≦3.0% | 110.0% |
| B1-60 | 0 point | <ref. I | 3.3 | Yes | 102.2 | <0.03 | 96.4 |
| | 1 month | <ref. I | 3.3 | Yes | 99.7 | <0.03 | 95.9 |
| | 2 months | <ref. I | 3.3 | Yes | 100.5 | <0.03 | 96.2 |
| | 3 months | <ref. I | 3.3 | Yes | 100.5 | <0.03 | 92.4 |
| | 6 months | <ref. I | 3.4 | Yes | 100.0 | <0.03 | 94.1 |
| B1-45 | 0 point | <ref. I | 3.3 | Yes | 101.3 | <0.03 | 95.8 |
| | 1 month | <ref. I | 3.3 | Yes | 98.7 | <0.03 | 94.0 |
| | 2 months | <ref. I | 3.3 | Yes | 100.7 | <0.03 | 95.7 |
| | 3 months | <ref. I | 3.3 | Yes | 100.3 | <0.03 | 91.6 |
| | 6 months | <ref. I | 3.3 | Yes | 100.8 | <0.03 | 91.8 |
| B2-45 | 0 point | <ref. I | 3.3 | Yes | 102.4 | <0.03 | 95.5 |
| | 1 month | <ref. I | 3.3 | Yes | 100.2 | <0.03 | 95.2 |
| | 2 months | <ref. I | 3.3 | Yes | 100.8 | <0.03 | 95.8 |
| | 3 months | <ref. I | 3.3 | Yes | 100.2 | <0.03 | 91.6 |
| | 6 months | <ref. I | 3.3 | Yes | 101.1 | <0.03 | 94.0 |
| B3-45 | 0 point | <ref. I | 3.3 | Yes | 101.9 | <0.03 | 95.3 |
| | 1 month | <ref. I | 3.3 | Yes | 100.0 | <0.03 | 95.3 |
| | 2 months | <ref. I | 3.3 | Yes | 100.4 | <0.03 | 95.4 |
| | 3 months | <ref. I | 3.3 | Yes | 100.4 | <0.03 | 92.0 |
| | 6 months | <ref. I | 3.3 | Yes | 100.7 | <0.03 | 93.2 |

Results are given as a mean of 3 determinations. As evident from Table 7, the composition solution remained clear under storage condition, no degradation of cabazitaxel and formation of impurity was observed, the pH of the solution remained unchanged. All these results indicate that the composition was stable. The additional contact with the stopper (inverted vial) as well did not alter the stability of the solution.

TABLE 8
Stability test of compositions of Examples 4 & 5-Storage 40° C./75% r.h. (vial upright)

| | | Tested Parameter | | | | | |
|---|---|---|---|---|---|---|---|
| | | | | Cabazitaxel | | | |
| | | | | identity | | | |
| | | | | retention | | | |
| | | | | time | Cabazitaxel | | |
| | | | pH | corresponds | 90.0-110.0% | Total | ethanol |
| Shelf life | | Clarity | 3.1- | to reference | (9.00-11.0 | Impurity | 90.0- |
| specification | | <ref. I | 3.7 | (±3%) | mg/ml) | ≦3.0% | 110.0% |
| B1-60 | 0 point | <ref. I | 3.3 | Yes | 102.2 | <0.03 | 96.4 |
| | 1 month | <ref. I | 3.3 | Yes | 97.0 | <0.03 | 96.3 |
| | 2 months | <ref. I | 3.3 | Yes | 100.1 | <0.03 | 96.3 |
| | 3 months | <ref. I | 3.3 | Yes | 100.5 | <0.03 | 92.9 |
| | 6 months | <ref. I | 3.3 | Yes | 100.0 | <0.03 | 94.4 |
| B1-45 | 0 point | <ref. I | 3.3 | Yes | 101.3 | <0.03 | 95.8 |
| | 1 month | <ref. I | 3.3 | Yes | 98.9 | <0.03 | 95.9 |
| | 2 months | <ref. I | 3.3 | Yes | 101.1 | <0.03 | 96.2 |
| | 3 months | <ref. I | 3.3 | Yes | 100.4 | <0.03 | 92.9 |
| | 6 months | <ref. I | 3.3 | Yes | 100.0 | <0.03 | 95.2 |
| B2-45 | 0 point | <ref. I | 3.3 | Yes | 102.4 | <0.03 | 95.5 |
| | 1 month | <ref. I | 3.3 | Yes | 98.6 | <0.03 | 95.4 |
| | 2 months | <ref. I | 3.3 | Yes | 101.7 | <0.03 | 95.8 |
| | 3 months | <ref. I | 3.3 | Yes | 100.6 | <0.03 | 92.5 |
| | 6 months | <ref. I | 3.4 | Yes | 100.5 | <0.03 | 93.7 |
| B3-45 | 0 point | <ref. I | 3.3 | Yes | 101.9 | <0.03 | 95.3 |
| | 1 month | <ref. I | 3.3 | Yes | 98.7 | <0.03 | 95.9 |
| | 2 months | <ref. I | 3.3 | Yes | 101.0 | <0.03 | 95.8 |
| | 3 months | <ref. I | 3.3 | Yes | 100.5 | <0.03 | 92.9 |
| | 6 months | <ref. I | 3.3 | Yes | 100.2 | <0.03 | 94.0 |

Results are given as a mean of 3 determinations. As evident from Table 8, the composition solution remained clear under storage condition, no degradation of cabazitaxel and formation of impurity was observed, the pH of the solution remained unchanged. All these results indicate that the composition was stable.

### Example 7

### Critical Micelle Concentration Analysis

The critical micelle concentration (CMC) of the infusion solutions prepared with cabazitaxel compositions can be investigated.

The intended cabazitaxel concentration of the final infusion solution should be about 0.10 to 0.26 mg/ml. Accordingly, the concentration of cabazitaxel studied in the infusion solutions is from about 0.10 to 0.26 mg/ml, in some embodiments 0.18 mg/ml. The infusion solution is preferably 0.9% NaCl or 5% glucose.

Micelle formation and decomplexation can be studied by the dye uptake method using 6-(p-Toluidino)-2-napthalenesulsonic acid sodium salt (TNS); see, e.g. Das and Hajra, Journal of Biological Chemistry (1992) 167(14): 9731. The theoretical critical micelle concentration polysorbate 80 in water is 12 μM at 25° C., which corresponds to 0.016 mg/ml; see, e.g Chou et al., J. Pharmaceutical Sciences (2005) 94(6):1368-1361.

To demonstrate measuring formation of micelle solutions of polysorbate 80 in 0.9% NaCl or 5% glucose ranging from 0.001 mg/ml to 55.556 mg/ml polysorbate 80 in each of the infusions are prepared. 90 μl of each sample is mixed in a 96 well plate with 10 μl of an appropriate TNS working solution (TNS concentration of 40 μM in each well). The fluorescence signal is measured at 355 nm excitation and 460 nm emission. By this method, in 0.9% NaCl or 5% glucose infusion solutions, the CMC of polysorbate 80 should be in the concentration range of from 9 to 18 μg/ml.

To demonstrate measuring decomplexation of micelles of polysorbate 80 by dilution in 0.9% NaCl or 5% Glucose Solution, a solution of polysorbate 80 in 0.9% NaCl and 5% glucose is further diluted to a polysorbate 80 concentration lower than the determined CMC. Polysorbate 80 solutions from 0.005 mg/ml to 6.094 mg/ml in 0.9% NaCl or 5% glucose are prepared. 90 μl of each sample is mixed with 10 μl of appropriate TNS working solution in a 96 well plate (TNS concentration of 40 μM in each well). The fluorescence signal is measured at 355 nm excitation and 460 nm emission.

The micelle formation of polysorbate 80 in human plasma can be studied. Solutions of polysorbate 80 in human plasma are prepared having concentrations of from 10 mg/ml to 0.005 mg/ml by diluting a stock polysorbate 80 solution (260.630 mg/ml PS80 in water) with human plasma. 90 μl of sample are transferred to a 96 well plate with 10 μl of the appropriate TNS working solution (TNS concentration of 40 μM in each well). The fluorescence signal is measured at 355 nm excitation and 460 nm emission.

Decomplexation of micelles of cabazitaxel formulation diluted in 0.9% NaCl, 5% Glucose or Human Plasma. Solutions with concentrations from 0.005 mg/ml to 6.094 mg/ml polysorbate 80 are prepared by dilution of the cabazitaxel formulation (10 mg/ml) to be assayed with infusion solution (0.9% NaCl or 5% glucose) or human plasma. 90 μl of each sample is transferred to a 96 well plate and mixed with 10 μl of the appropriate TNS working solution (TNS concentration of 40 μM in each well). The fluorescence signal is measured at 355 nm excitation and 460 nm emission. Under these conditions, the micelles should decomplex below a concentration of polysorbate 80 of about 45 μg/ml, in some embodiments 18 μg/ml, in some embodiments between 9 to 18 μg/ml.

Infusion Simulation. Generally, the infusion solution (254.5 ml when using 4.5 ml of the formulation of Example A and B) is infused during one hour. Assuming that 254.5 ml of the infusion solution prepared with the formulation of Example A and B are infused into 3000 ml human plasma (final concentration of cabazitaxel 0.138 mg/ml), the concentrations of cabazitaxel and polysorbate 80 are estimated to be as follows:

| | | | Final | | |
|---|---|---|---|---|---|
| | Volume | | volume | | Approximate |
| Time | Infused | Cabazitaxel | (infusion + | Cabazitaxel | PS80 |
| (min) | (ml) | Infused (mg) | 3L plasma) | (mg/ml) | (mg/ml) |
| 15 | 63.63 | 11.25 | 3063.63 | 0.0037 | 0.0955 |
| 30 | 127.25 | 22.50 | 3127.50 | 0.0072 | 0.1871 |
| 45 | 190.88 | 33.75 | 3190.88 | 0.0106 | 0.2750 |
| 60 | 254.5 | 45 | 3254.5 | 0.0138 | 0.3595 |

Solutions including these estimated concentrations of cabazitaxel and polysorbate 80 are prepared from the cabazitaxel formulations (10 mg/ml) by diluting with human plasma. The same dilutions of the placebo (comprising no cabazitaxel) solution are also prepared in human plasma.

In-Use Study (Mimic Infusion Conditions): The infusion conditions can be mimicked to show that the micelle system is stable over the infusion time of 60 minutes. The prepared infusion solutions are filled into infusion bags equipped with infusion sets. The infusion sets are opened and samples are withdrawn over a period of 60 minutes. Samples are transferred to a 96 well plate and 10 μl of the appropriate TNS working solution is added. (Final TNS concentration 40 μM.) The fluorescence signal is measured at 355 nm excitation and 460 nm emission.

The foregoing is illustrative of the present invention, and is not to be construed as limiting thereof. The invention is defined by the following claims, with equivalents of the claims to be included therein.

1. A pharmaceutical composition comprising:
a) from 0.5 to 1.5 weight-% of cabazitaxel;
b) from 20 to 40 weight-% of a polysorbate;
c) from 15 to 22 weight-% of a C₂-C₄ alcohol;
d) from 0.3 to 0.6 weight-% of an organic acid;
e) from 40 to 60 weight-% of a polyethylene glycol; and
f) from 0 to 5 weight-% of water;
in each case based on the total weight of the composition.

2. The composition of claim 1, comprising:
a) from 0.92 to 1.01 weight-% of the cabazitaxel;
b) from 23 to 27 weight-% of the polysorbate;
c) from 17 to 21 weight-% of the C₂-C₄ alcohol;
d) from 0.4 to 0.5 weight-% of the organic acid;
e) from 50 to 57 weight-% of the polyethylene glycol; and
f) from 0 to 1 weight-% of water;
in each case based on the total weight of the composition.

3. The composition of claim 1, wherein at least 99 weight-% of the composition consists of the components a), b), c), d), and e).

4. The composition of claim 1, wherein the composition is liquid at a temperature in the range of from 24 to 26° C. and a pressure in the range of from 0.95 to 1.05 bar.

5. The composition of claim 1, wherein the polysorbate is polyoxyethylene(20) sorbitan monooleate, the C₂-C₄ alcohol is absolute ethanol, the organic acid according to d) is anhydrous citric acid, and the polyethylene glycol is polyethylene glycol 300.

6. The composition of claim 2, wherein the polysorbate is polyoxyethylene(20) sorbitan monooleate, the C₂-C₄ alcohol is absolute ethanol, the organic acid is anhydrous citric acid, and the polyethylene glycol is polyethylene glycol 300.

7. The composition of claim 1, having a pH in the range of from 2 to 4, wherein the pH relates to the pH of the composition diluted with purified water at a ratio of composition relative to water of 1:9, the pH value being determined with a pH meter comprising a pH sensitive glass electrode at a temperature of the diluted composition in the range of from 20 to 25° C.

8. The composition of claim 7, having a pH in the range of from 2.8 to 3.5.

9. The composition of claim 6, having a pH in the range of from 2 to 4, wherein the pH relates to the pH of the composition diluted with purified water at a ratio of composition relative to water of 1:9, the pH value being determined with a pH meter comprising a pH sensitive glass electrode at a temperature of the diluted composition in the range of from 20 to 25° C.

10. The composition of claim 9, having a pH in the range of from 2.8 to 3.5.

11. The composition of claim 6, obtained by a process comprising:
(i) dissolving the organic acid in the C₂-C₄ alcohol, obtaining a mixture I;
(ii) dissolving the cabazitaxel monohydrate or anhydrous cabazitaxel in the mixture I, obtaining a mixture II;
(iii) dissolving the polysorbate in the mixture II, obtaining a mixture III;
(iv) adding the polyethylene glycol to the mixture III, obtaining a mixture IV;
(v) mixing the mixture IV, obtaining the mixture V; and
(vi) purging the mixture of V with nitrogen, obtaining the composition;
wherein the composition obtained after step (vi) comprises less than 3% dissolved oxygen.

12. The composition of claim 2, comprised in a vial.

13. The composition of claim 6, comprised in a vial, wherein the vial comprises from 42 to 48 mg of the cabazitaxel.

14. The composition of claim 13, wherein the vial is an air-tight vial comprising an air-tight stopper.

15. The composition of claim 14, wherein the headspace of the vial comprises less than 1% oxygen.

16. The composition of claim 13, comprising less than 5% dissolved oxygen.

17. The composition of claim 6, comprised in a vial, wherein the vial comprises from 57 to 63 mg of the cabazitaxel.

18. The composition of claim 17, wherein the vial is an air-tight vial comprising an air-tight stopper.

19. The composition of claim 18, wherein the headspace of the vial comprises less than 1% oxygen.

20. The composition of claim 18, comprising less than 5% dissolved oxygen.

21. A one-vial pharmaceutical composition comprising:
a) from 9.5 to 10.5 mg/ml of cabazitaxel;
b) from 238 to 280 mg/ml of polyoxyethylene (20) sorbitan monooleate;
c) from 176 to 218 mg/ml of absolute ethanol;
d) from 4 to 5 mg/ml of anhydrous citric acid;
e) from 520 to 590 mg/ml polyethylene glycol 300; and
f) from 0 to 1 mg/ml of water,
wherein the one-vial pharmaceutical composition has a total volume of at least 4.5 ml.

22. The one-vial pharmaceutical composition of claim 21, comprising an air-tight vial comprising an air-tight stopper.

23. The one-vial pharmaceutical composition of claim 22, wherein the headspace of the vial comprises less than 1% oxygen.

24. The one-vial pharmaceutical composition of claim 22, comprising less than 5% dissolved oxygen.

25. The one-vial pharmaceutical composition of claim 21, wherein the total volume is 4.6 to 5.4 ml.

26. The one-vial pharmaceutical composition of claim 21, wherein the total volume is 6.2 to 7.0 ml.

27. A parenteral formulation comprising the pharmaceutical composition according to claim 6 dissolved in a parenteral fluid selected from a saline solution or a glucose solution, wherein the parenteral formulation comprises cabazitaxel at a concentration of from 0.1 to 0.26 mg/ml.

28. A method for treating prostate cancer comprising administering a parenteral formulation according to claim 27 to a patient in need thereof.

29. The method of claim 28, further comprising administering the parenteral formulation in combination with prednisone or prednisolone.
